(19) United States
(12) Patent Application Publication
(10) Pub. No.: US 2006/0222601 A1
(43) Pub. Date: Oct. 5, 2006

(54) Oral care compositions with color changing indicator

(76) Inventors: Ram W. Sabnis, Eagan, MN (US); Timothy D. Kehoe, St. Paul, MN (US); Robert James Balchunis, St. Paul, MN (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/391,671

(22) Filed: Mar. 28, 2006

Related U.S. Application Data
(60) Provisional application No. 60/696,872, filed on Jul. 6, 2005.
(60) Provisional application No. 60/711,183, filed on Aug. 25, 2005.
(60) Provisional application No. 60/734,219, filed on Nov. 7, 2005.
(60) Provisional application No. 60/763,708, filed on Jan. 31, 2006.
(60) Provisional application No. 60/666,028, filed on Mar. 29, 2005.

(51) Int. Cl. A61K 8/368; A61K 8/46

(52) U.S. Cl. 424/49

(57) ABSTRACT

The invention describes color changing toothpastes and mouthwashes which contains acid-base indicator(s) for interaction with the oral cavity to provide a color change indicative of treatment time.

### CROSS REFERENCE TO RELATED APPLICATIONS

This application claims benefit under 35 U.S.C. § 119(e) to U.S. Ser. Nos. 60/696,872, filed Jul. 6, 2005 (Attorney docket number 186573/US), entitled “Color Changing Compositions and Articles”, 60/711,183, filed Aug. 25, 2005 (Attorney docket number 186978/US), entitled “Substituted Phenol-Based Aqueous Indicators”.

This application also claims benefit under 35 U.S.C. § 119(e) to U.S. Ser. Nos. 60/734,219, filed Nov. 7, 2005 (Attorney docket number 187222/US), entitled “Color Changing Toothpaste” and 60/763,708, filed Jan. 31, 2006 (Attorney docket number 187482/US), entitled “Mouthwash Composition”, the contents of which are incorporated herein by reference in their entirety.

### FIELD OF THE INVENTION

The invention relates generally oral care compositions that have an acid-based indicator that is pH sensitive. The colored oral care composition can change color from colored to clear, clear to colored or from a first color to a second color dependent up on the choice of acid-base indicator(s).

### BACKGROUND OF THE INVENTION

As is known, inducing children (and adults to some extent) to brush their teeth on a regular basis presents a difficult challenge. The brushing of teeth is perceived as a bothersome necessity by many adults and even more so by children. Insofar as children are concerned, the problem is exacerbated by the fact that children are highly sensitive to bitter tastes, possess a heightened gag reflex and typically utilize an equal amount of toothpaste as adults while having a mouth that is one fourth the size of the adult mouth. Thus, not only is brushing of the teeth an uncomfortable experience for children, but additionally a child's lack of appreciation of the benefits of regular brushing coupled with a child's short attention span renders the twice daily brushing regimen devoid of any positive reinforcement for the typical child.

The availability of a toothpaste or dentrifice which would make brushing more enjoyable for children would provide an inducement lacking in existing toothpaste and dentrifice formulations. A toothpaste which produces a dynamic color change, has a reduced bitter taste and less of the annoying foaming action that often chokes children's small mouths would permit the accomplishment of basic oral hygiene with improved results and less aggravation. In the past, efforts have been made to develop toothpaste formulations which undergo a color change upon brushing.

The formation of dental plaque leads to dental caries, gingival inflammation, periodontal disease, and eventually tooth loss. Dental plaque is a mixture of bacteria, epithelial cells, leukocytes, macrophages, and other oral exudate. Said bacteria produce highly branched polysaccharides which together with microorganisms from the oral cavity form an adhesive matrix for the continued proliferation of plaque.

Generally an individual gargles or swishes the mouthwash for a given period of time to treat a sore throat, to reduce bacteria, and/or to reduce or eliminate bad breath. There is no way to know when an appropriate time period has passed so that the individual knows that sufficient treatment of the mouthwash has occurred.

There remains a need for improved mouthwash and toothpaste compositions, particularly for use by children, which can be readily formulated, can produce a dynamic color change during use and/or which reduce objectionable bacteria in the mouth during contact.

### BRIEF SUMMARY OF THE INVENTION

Among the several features of the invention may be noted the provision of novel oral care compositions such as mouthwash and toothpaste compositions containing an acid-base indicator(s) that is pH and/or time sensitive to provide a color change indicative of contact time. The compositions can provide a color change that occurs independently of the pH in the mouth or that can occur with a change in pH during contact with the mouth. The composition(s) can be readily formulated from available materials.

The present invention is directed to a oral care compositions, including but not limited to mouthwashes, toothpastes/gels containing acid-base indicators for interaction to provide a color change upon treatment with the oral cavity, the acid-based indicators comprising those described throughout the application vide infra.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### DETAILED DESCRIPTION

In accordance with the present invention, it has now been found that a color-changing oral care composition, i.e, toothpaste or mouthwash, can be formulated by incorporating acid-base indicators into the composition of interest to provide a color change indicative of appropriate contact time. By producing a dynamic color change (from colorless to clear, from one color to another color, or from color to clear) after a predetermined contact time, the compositions of the present invention advantageously makes basic oral hygiene more appealing, less aggravating and more effective especially for children.

Suitable acid-base indicators used in the present invention are generally colored under basic condition and change color or fade to clear in non-basic condition. Acid-base indicators which are colored on alkaline pH side (pH>7) and turn clear on acidic pH (pH<7) are most useful. Typically, the acid-base indicators are colored at pH between about 9 and 10, and turn clear at pH between about 6 and 8.

Representative examples of acid-base indicators useful in the compositions of the present invention include, but are not limited to, picric acid, matius yellow, 2,6-dinitrophenol, 2,4-dinitrophenol, phenacetolin, 2,5-dinitrophenol, isopicramic acid, o-nitrophenol, m-nitrophenol, p-nitrophenol, 6,8-dinitro-2,4-(1H,3H)quinazolinedione, nitroamine, ethyl bis(2,4-dinitrophenyl)-acetate, 2,4,6-trinitrotoluene, 1,3,5-trinitrobenzene, 2,4,6-tribromobenzoic acid, 2-(p-dimethylaminophenyl)azopyridine, metanil yellow, p-methyl red, 4-phenylazodiphenylamine, benzopurpurin 4B, tropaeolin OO, fast garnet GBC base, alizarin yellow R, benzyl orange, m-methyl red, 4-(m-tolyl)-azo-N,N-dimethyl-aniline, oil yellow II, methyl orange, ethyl orange, hessian purple N, congo red, N-pnehyl-1-naphthyl-aminoazobenzene-p-sulfonic acid, 4-(4′-dimethylamino-1′-naphthyl)-azo-3-methoxy-benzenesulfonic acid, p-ethoxychrysoidine, α-naphthyl red, chrysoidine, 1-naphthylaminoazobenzene-p-sulfonic acid, methyl red, 2-(p-dimethylaminophenyl)-azopyridine, ethyl red, propyl red, N-phenyl-1-naphthyl-aminoazo-o-carboxybenzene, nitrazol yellow, brilliant yellow, brilliant yellow S, orange II, propyl-o-naphthyl orange, orange I, orange IV, hessian, Bordeaux, diazo violet, α-naphthol violet, alizarin yellow GG, chrome orange GR, sulfone acid blue R, lanacyl violet BF, tropaeolin O, orange G, crystal violet, methyl violet B, malachite green, brilliant green, ethyl violet, methyl violet 6B, ethyl/methyl green, basic fuchsine, acid, fuchsine, patent blue V, alkali blue, aniline blue, o-naphthol benzein, pentamethoxy red, hexamethoxy red, tetrabromophenolphthalein ethyl ester K salt, tetraiodophenolsulfophthlein, bromochlorophenol blue, bromocresol green, chlorocresol green, chlorophenol red, bromocresol purple, sulfonaphthyl red, bromophenol red, dibromophenol-tetrabromophenol-sulfophthlein, bromothymol blue, aurin, phenol red, o-cresol benzein, o-cresol red, α-naphtholphthlein, m-cresol purple, p-xylenol blue, thymol blue, phenoltetrachlorophthlein, o-cresolphthalein, α-naphtholbenzein, phenoltetraiodophthlein, phenolphthalein, thymolphthlein, eosin Y, erythrosine B, erythrosine, galleon, brilliant cresyl blue, resazurin, lacmoid, litmus, azolitmus, azolitmin, neutral red, nile blue 2B, nile blue A, hematoxylin, quinaldine red, pinachrome, indo-oxine, quinoline blue, bis-5-bromovanillidenecyclohexanone, bis-(2′-hydroxystyryl)ketone, curcumin, bis-(4-hydroxy-3-ethoxy-benzylidene)-cyclohexanone, thiazole yellow G, alizarin blue B, alizarin red S, carminic acid, alizarin orange, alizarin, rufianic acid, rufianic blue, alizarin blue SWR, and indigocarmine.

With the suitable selection of acid-base indicators, it is possible to produce any color. The acid-base indicators are preferably in the form of a salt, such as a sodium salt generated by reacting the indicator with sodium hydroxide, so as to permit its solubilization into the present composition. Additionally, combinations of two or more indicators may be used.

Acid-base indicators are usually effective when present in small amounts in the compositions of the invention but generally are present in amounts from about 0.01% up to about 20% by weight, from about 0.5% to about 10% by weight and from about 0.8% to about 8% by weight of the total weight of the composition.

Selection of an appropriate basic material is important for color change of acidic dye indicators in the colored compositions of the present invention. Desirable basic reagents, which should readily volatilize at ambient temperatures for use in the present compositions, include, but are not limited to, aminoalcohols, such as alkylamines, such as methylamine, dimethylamine, ethylamine, diethylamine, triethylamine, ethyleneamine, diethyleneamine, morpholine, ammonia, triethanolamine. Other amines or amino alcohols are suitable provided they are non-toxic.

The selection of the kind and the amount of basic reagent used enables control of fading time of the color after application. Suitable basic reagents which readily volatilize at ambient temperatures, typically have a vapor pressure higher than about 10 mm Hg at 20° C. The selection of the base also depends on solubility in water, toxicity and odor. Therefore, aminoalcohols useful in the compositions of the present invention include, but are not limited to triethanolamine (TEA) and/or diethylamine. TEA, for example, is clear, non-toxic and does not emit a noxious odor.

The basic reagent(s) is generally present in the composition of the invention in an amount from about 0.01% up to about 20% by weight, from about 0.2% to about 10% by weight and from about 0.5% to about 5% by weight.

It should be understood that the term “comprising” (or comprises) includes the more restrictive terms consisting of and consisting essentially of.

Particular phthaleins useful in the invention have the formula (I):

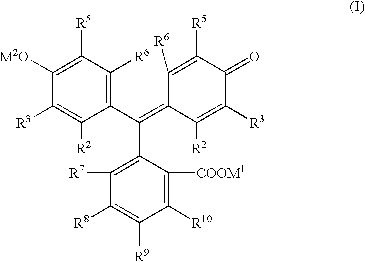

wherein R², R³, R⁵, R⁶, R⁷, R⁸, R⁹ and R¹⁰ are each, independently of one another, selected from the group consisting of hydrogen, —OH, —SH, —CN, —NO₂, halo, fluoro, chloro, bromo, iodo, lower alkyl, substituted lower alkyl, lower heteroalkyl, substituted lower heteroalkyl, cycloalkyl, substituted cycloalkyl, cycloheteroalkyl, substituted cycloheteroalkyl, lower haloalkyl, monohalomethyl, dihalomethyl, trihalomethyl, trifluoromethyl, lower alkylthio, substituted lower alkylthio, lower alkoxy, substituted lower alkoxy, methoxy, substituted methoxy, lower heteroalkoxy, substituted lower heteroalkoxy, cycloalkoxy, substituted cycloalkoxy, cycloheteroalkoxy, substituted cycloheteroalkoxy, lower haloalkoxy, monohalomethoxy, dihalomethoxy, trihalomethoxy, trifluoromethoxy, amino, lower di- or monoalkylamino, substituted lower di- or monoalkylamino, aryl, substituted aryl, aryloxy, substituted aryloxy, phenoxy, substituted phenoxy, arylalkyl, substituted arylalkyl, arylalkyloxy, substituted arylalkyloxy, benzyl, benzyloxy, heteroaryl, substituted heteroaryl, heteroaryloxy, substituted heteroaryloxy, heteroarylalkyl, substituted heteroarylalkyl, heteroarylalkyloxy, substituted heteroarylalkyloxy, carboxyl, lower alkoxycarbonyl, substituted lower alkoxycarbonyl, aryloxycarbonyl, substituted aryloxycarbonyl, arylalkyloxycarbonyl, substituted arylalkyloxycarbonyl, carbamate, substituted carbamate, carbamoyl, substituted carbamoyl, sulfamoyl or substituted sulfamoyl.

Alternatively, R² and R³, R⁵ and R⁶ or R² and R³, and R⁵ and R⁶ can form cyclic ring structures that are heterocyclic, heteroaromatic, aromatic or nonaromatic and can contain one or more heteroatoms to form, for example, a quinoline, napthalene, etc.

Additionally, R⁷ and R⁸, R⁸ and R⁹, R⁹ and R¹⁰ or combinations thereof can form cyclic ring structures that are heterocyclic, heteroaromatic, aromatic or nonaromatic and can contain one or more heteroatoms to form, for example, a quinoline, napthalene, etc.

Optionally, one of the carbons connected to R², R³, R⁵ or R⁶ can be substituted with a nitrogen atom.

M¹ and M² are each independently a hydrogen atom, a metal ion or an ammonium ion.

In certain embodiments, R² is selected from the group consisting of hydrogen, nitro, amino and alkyl; R³ is selected from the group consisting of hydrogen, phenyl, alkyl, nitro, acetamido and alkoxy; R⁵ is selected from the group consisting of hydrogen, halo, and alkyl; and R⁶ is selected from the group consisting of hydrogen and alkyl.

In certain other embodiments, R² is selected from the group consisting of hydrogen and methyl; R³ is selected from the group consisting of hydrogen, phenyl, isopropyl, methyl, ethyl, sec-butyl, nitro and methoxy; R⁵ is selected from the group consisting of hydrogen, bromo, methoxy, isopropyl and methyl; and R⁶ is selected from the group consisting of hydrogen and methyl.

In other embodiments, R² is hydrogen, R³is Me, and R⁵, R⁶, R⁷, R⁸, R⁹ and R¹⁰ are all hydrogen atoms, or R² is Me, R³is a hydrogen atom, R⁵ is an iso-propyl group and R⁶, R⁷, R⁸, R⁹ and R¹⁰ are all hydrogen atoms, or R² is H, R³is Me, R⁵ is Br and R⁶, R⁷, R⁸, R⁹ and R¹⁰ are all hydrogen atoms, or R² is Me, R³ is Br, R⁵is an isopropyl and R⁶, R⁷, R⁸, R⁹ and R¹⁰ are all hydrogen atoms. In certain embodiments, one or more of these compounds may be excluded from certain aspects of the invention.

In still other embodiments, R² is H, R³ is phenyl and R⁵, R⁶, R⁷, R⁸, R⁹ and R¹⁰ are all hydrogen atoms, or R² is H, R³ and R⁵ are isopropyl and R⁶, R⁷, R⁸, R⁹ and R¹⁰ are all hydrogen atoms, or R² is H, R³ is methyl, R⁵ is H, R⁶ is methyl, R⁷, R⁸, R⁹ and R¹⁰ are all hydrogen atoms, or R² is H, R³ and R⁵ are methoxy and R⁶, R⁷, R⁸, R⁹ and R¹⁰ are all hydrogen atoms, or R² is H, R³ and R⁵ are methyl and R⁶, R⁷, R⁸, R⁹ and R¹⁰ are all hydrogen atoms, or R² is H, R³ is ethyl and R⁵, R⁶, R⁷, R⁸, R⁹ and R¹⁰ are all hydrogen atoms, or R² is H, R³ is isopropyl and R⁵, R⁶, R⁷, R⁸, R⁹ and R¹⁰ are all hydrogen atoms, or R² is H, R³ is methoxide and R⁵, R⁶, R⁷, R⁸, R⁹ and R¹⁰ are all hydrogen atoms, or R², R³ and R⁵ are all methyl and R⁶, R⁷, R⁸, R⁹ and R¹⁰ are all hydrogen atoms, or R², R³, R⁵, R⁶, R⁷, R⁸, R⁹, R¹⁰ are all hydrogen atoms and R³ is sec-butyl, or R², R³, R⁵, R⁶, R⁷, R⁸, R⁹, R¹⁰ are all hydrogen atoms and R³ is nitro.

In one aspect, the compound where R², R³, R⁵, R⁶, R⁷, R⁸, R⁹ and R¹⁰ are all hydrogen atoms is excluded from the compositions.

In particular, at least one of M¹ or M² is a metal or ammonium ion.

It should be understood, that the salt form of the indicator can be isolated prior to use or prepared in situ. Ideally, the salt is formed as a mono-salt or a di-salt, meaning that excess base is not present and either 1 or 2 equivalents of base react with the acidic protons of the indicator.

The following table provides phthaleins of particular interest.

| R² | R³ | R⁵ | R⁶ | Color |
|---|---|---|---|---|
| H | phenyl | H | H | purple |
| H | i-propyl | i-propyl | H | violet |
| H | Me | H | Me | blue |
| H | OMe | OMe | H | teal |
| H | Me | Me | H | purple |
| H | Et | H | H | magenta |
| H | i-propyl | H | H | pink |
| H | OMe | H | H | blue |
| Me | Me | Me | H | teal |
| H | Me | H | H | magenta |
| H | i-propyl | H | Me | blue |
| H | Me | Br | H | purple |
| H | i-propyl | Br | Me | teal |
| H | sec-butyl | H | H | pink |
| H | NO₂ | H | H | yellow |

In another aspect, the acid-base indicator can be a substituted phenol of formula (II):

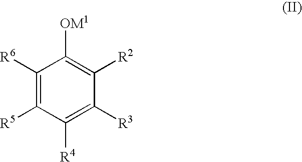

wherein R², R³, R⁵, R⁶ and M¹ are as defined above and R⁴ is selected from the same group as R², R³, R⁵ and R⁶.

Alternatively, R² and R³, R³ and R⁴, R⁴ and R⁵, or R⁵ and R⁶ can form cyclic ring structures that are heterocyclic, heteroaromatic, aromatic or nonaromatic and can contain one or more heteroatoms to form, for example, a quinoline, napthalene, etc.

In one aspect, one or more of R² through R⁶, independently, is a nitro (—NO₂) group and the remaining R groups are selected from those provided above.

Additionally, substituted hydrazides are useful in the compositions of the invention and can have one of two formulae:

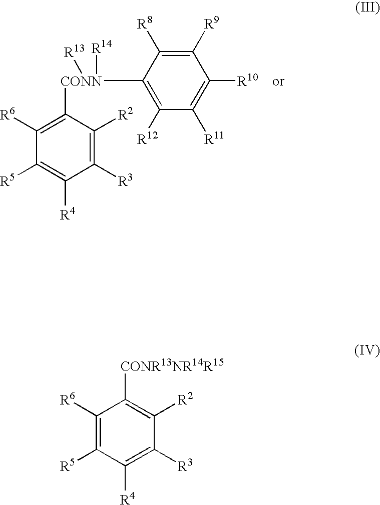

wherein R² through R⁶ are as defined above and R⁸ through R¹² are the same substituents as R² through R⁶. R¹³, R¹⁴ and R¹⁵ (if present) are each, independently of one another, a hydrogen atom, an alkyl group, a substituted alkyl group, any aryl group or a substituted aryl group.

In certain embodiments for compound formulae (II), R¹³ and R¹⁴ are hydrogen atoms and for compound formulae (III), R¹³, R¹⁴ and R¹⁵ are all hydrogen atoms.

In certain aspects, compounds of formulae (III) can have one or more hydroxyl groups, which can be deprotonated to form a salt. For example, formulae (IIIa) provides one isomer where a hydroxyl is present at the R² position as a salt. M² is as defined above for M¹. It should be understood that one or more of R² through R¹² could have a hydroxyl at that given position, and that hydroxyl could be in a salt form.

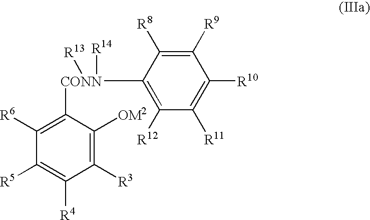

“Alkyl,” by itself or as part of another substituent, refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. Preferably, an alkyl group comprises from 1 to 15 carbon atoms (C₁-C₁₅ alkyl), more preferably from 1 to 10 carbon atoms (C₁-C₁₀ alkyl) and even more preferably from 1 to 6 carbon atoms (C₁-C₆ alkyl or lower alkyl).

“Alkanyl,” by itself or as part of another substituent, refers to a saturated branched, straight-chain or cyclic alkyl radical derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkenyl,” by itself or as part of another substituent, refers to an unsaturated branched, straight-chain or cyclic alkyl radical having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl,” by itself or as part of another substituent refers to an unsaturated branched, straight-chain or cyclic alkyl radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Alkyldiyl” by itself or as part of another substituent refers to a saturated or unsaturated, branched, straight-chain or cyclic divalent hydrocarbon group derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center can form bonds with the same or different atoms. Typical alkyldiyl groups include, but are not limited to, methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. Where specific levels of saturation are intended, the nomenclature alkanyldiyl, alkenyldiyl and/or alkynyldiyl is used. Where it is specifically intended that the two valencies are on the same carbon atom, the nomenclature “alkylidene” is used. In preferred embodiments, the alkyldiyl group comprises from 1 to 6 carbon atoms (C1-C6 alkyldiyl). Also preferred are saturated acyclic alkanyldiyl groups in which the radical centers are at the terminal carbons, e.g., methandiyl (methano); ethan-1,2-diyl (ethano); propan-1,3-diyl (propano); butan-1,4-diyl (butano); and the like (also referred to as alkylenos, defined infra).

“Alkyleno,” by itself or as part of another substituent, refers to a straight-chain saturated or unsaturated alkyldiyl group having two terminal monovalent radical centers derived by the removal of one hydrogen atom from each of the two terminal carbon atoms of straight-chain parent alkane, alkene or alkyne. The locant of a double bond or triple bond, if present, in a particular alkyleno is indicated in square brackets. Typical alkyleno groups include, but are not limited to, methano; ethylenos such as ethano, etheno, ethyno; propylenos such as propano, prop[1]eno, propa[1,2]dieno, prop[1]yno, etc.; butylenos such as butano, but[1]eno, but[2]eno, buta[1,3]dieno, but[1]yno, but[2]yno, buta[1,3]diyno, etc.; and the like. Where specific levels of saturation are intended, the nomenclature alkano, alkeno and/or alkyno is used. In preferred embodiments, the alkyleno group is (C1-C6) or (C1-C3) alkyleno. Also preferred are straight-chain saturated alkano groups, e.g., methano, ethano, propano, butano, and the like.

“Alkoxy,” by itself or as part of another substituent, refers to a radical of the formula —OR, where R is an alkyl or cycloalkyl group as defined herein. Representative examples alkoxy groups include, but are not limited to, methoxy, ethoxy, propoxy, isopropoxy, butoxy, tert-butoxy, cyclopropyloxy, cyclopentyloxy, cyclohexyloxy and the like.

“Alkoxycarbonyl,” by itself or as part of another substituent, refers to a radical of the formula —C(O)-alkoxy, where alkoxy is as defined herein.

“Alkylthio,” by itself or as part of another substituent, refers to a radical of the formula —SR, where R is an alkyl or cycloalkyl group as defined herein. Representative examples of Alkylthio groups include, but are not limited to, methylthio, ethylthio, propylthio, isopropylthio, butylthio tert-butylthio, cyclopropylthio, cyclopentylthio, cyclohexylthio, and the like.

“Aryl,” by itself or as part of another substituent, refers to a monovalent aromatic hydrocarbon group derived by the removal of one hydrogen atom from a single carbon atom of a parent aromatic ring system, as defined herein. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. Preferably, an aryl group comprises from 6 to 20 carbon atoms (C₆-C₂₀ aryl), more preferably from 6 to 15 carbon atoms (C₆-C₁₅ aryl) and even more preferably from 6 to 10 carbon atoms (C₆-C₁₀ aryl).

“Arylalkyl,” by itself or as part of another substituent, refers to an acyclic alkyl group in which one of the hydrogen atoms bonded to a carbon atom, typically a terminal or sp³ carbon atom, is replaced with an aryl group as, as defined herein. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. Preferably, an arylalkyl group is (C₆-C₃₀) arylalkyl, e.g., the alkanyl, alkenyl or alkynyl moiety of the arylalkyl group is (C₁-C₁₀) alkyl and the aryl moiety is (C₆-C₂₀) aryl, more preferably, an arylalkyl group is (C₆-C₂₀) arylalkyl, e.g., the alkanyl, alkenyl or alkynyl moiety of the arylalkyl group is (C₁-C₈) alkyl and the aryl moiety is (C₆-C₁₂) aryl, and even more preferably, an arylalkyl group is (C₆-C₁₅) arylalkyl, e.g., the alkanyl, alkenyl or alkynyl moiety of the arylalkyl group is (C₁-C₅) alkyl and the aryl moiety is (C₆-C₁₀) aryl.

“Aryloxy,” by itself or as part of another substituent, refers to a radical of the formula —O-aryl, where aryl is as defined herein.

“Arylalkyloxy, by itself or as part of another substituent, refers to a radical of the formula —O-arylalkyl, where arylalkyl is as defined herein.

“Aryloxycarbonyl,” by itself or as part of another substituent, refers to a radical of the formula —C(O)—O-aryl, where aryl is as defined herein.

“Carbamoyl,” by itself or as part of another substituent, refers to a radical of the formula —C(O)NR′R″, where R′ and R″ are each, independently of one another, selected from the group consisting of hydrogen, alkyl and cycloalkyl as defined herein, or alternatively, R′ and R″, taken together with the nitrogen atom to which they are bonded, form a 5-, 6- or 7-membered cycloheteroalkyl ring as defined herein, which may optionally include from 1 to 4 of the same or different additional heteroatoms selected from the group consisting of O, S and N.

“Compounds of the invention” refers to compounds encompassed by the various descriptions and structural formulae disclosed herein. The compounds of the invention may be identified by either their chemical structure and/or chemical name. When the chemical structure and chemical name conflict, the chemical structure is determinative of the identity of the compound. The compounds of the invention may contain one or more chiral centers and/or double bonds and therefore may exist as stereoisomers, such as double-bond isomers (i.e., geometric isomers), rotamers, enantiomers or diastereomers. Accordingly, when stereochemistry at chiral centers is not specified, the chemical structures depicted herein encompass all possible configurations at those chiral centers including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds of the invention may also exist in several tautomeric forms including the enol form, the keto form and mixtures thereof. Accordingly, the chemical structures depicted herein encompass all possible tautomeric forms of the illustrated compounds. The compounds of the invention may also include isotopically labeled compounds where one or more atoms have an atomic mass different from the atomic mass conventionally found in nature. Examples of isotopes that may be incorporated into the compounds of the invention include, but are not limited to, ²H, ³H, ¹¹C, ¹³C, ¹⁴C, ¹⁵N, ¹⁸O, ¹⁷O, ³¹P, ³²P, 35S, ¹⁸F and ³⁶Cl. Compounds of the invention may exist in unsolvated forms as well as solvated forms, including hydrated forms and as N-oxides. In general, the hydrated, solvated and N-oxide forms are within the scope of the present invention. Certain compounds of the present invention may exist in multiple crystalline or amorphous forms. In general, all physical forms are equivalent for the uses contemplated by the present invention and are intended to be within the scope of the present invention.

“Cycloalkyl,” by itself or as part of another substituent, refers to a saturated or unsaturated cyclic alkyl radical, as defined herein. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane, and the like. Preferably, the cycloalkyl group comprises from 3 to 10 ring atoms (C₃-C₁₀ cycloalkyl) and more preferably from 3 to 7 ring atoms (C₃-C₇ cycloalkyl).

“Cycloheteroalkyl,” by itself or as part of another substituent, refers to a saturated or unsaturated cyclic alkyl radical in which one or more carbon atoms (and optionally any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidone, quinuclidine, and the like. Preferably, the cycloheteroalkyl group comprises from 3 to 10 ring atoms (3-10 membered cycloheteroalkyl) and more preferably from 5 to 7 ring atoms (5-7 membered cycloheteroalkyl).

A cycloheteroalkyl group may be substituted at a heteroatom, for example, a nitrogen atom, with a lower alkyl group. As specific examples, N-methyl-imidazolidinyl, N-methyl-morpholinyl, N-methyl-piperazinyl, N-methyl-piperidinyl, N-methyl-pyrazolidinyl and N-methyl-pyrrolidinyl are included within the definition of “cycloheteroalkyl.” A cycloheteralkyl group may be attached to the remainder of the molecule via a ring carbon atom or a ring heteroatom.

“Dialkylamino” or “Monoalkylamino,” by themselves or as part of other substituents, refer to radicals of the formula —NRR and —NHR, respectively, where each R is independently selected from the group consisting of alkyl and cycloalkyl, as defined herein. Representative examples of dialkylamino groups include, but are not limited to, dimethylamino, methylethylamino, di-(1-methylethyl)amino, (cyclohexyl)(methyl)amino, (cyclohexyl)(ethyl)amino, (cyclohexyl)(propyl)amino and the like. Representative examples of monalkylamino groups include, but are not limited to, methylamino, ethylamino, propylamino, isopropylamino, cyclohexylamino, and the like.

“Halogen” or “Halo,” by themselves or as part of another substituent, refer to a fluoro, chloro, bromo and/or iodo radical.

“Haloalkyl,” by itself or as part of another substituent, refers to an alkyl group as defined herein in which one or more of the hydrogen atoms is replaced with a halo group. The term “haloalkyl” is specifically meant to include monohaloalkyls, dihaloalkyls, trihaloalkyls, etc. up to perhaloalkyls. The halo groups substituting a haloalkyl can be the same, or they can be different. For example, the expression “(C₁-C₂) haloalkyl” includes 1-fluoromethyl, 1-fluoro-2-chloroethyl, difluoromethyl, trifluoromethyl, 1-fluoroethyl, 1,1-difluoroethyl, 1,2-difluoroethyl, 1,1,1-trifluoroethyl, perfluoroethyl, etc. “Haloalkyloxy,” by itself or as part of another substituent, refers to a group of the formula —O-haloalkyl, where haloalkyl is as defined herein.

“Heteroalkyl,” “Heteroalkanyl,” “Heteroalkenyl,” “Heteroalkynyl,” “Heteroalkyldiyl” and “Heteroalkyleno,” by themselves or as part of other substituents, refer to alkyl, alkanyl, alkenyl, alkynyl, alkyldiyl and alkyleno groups, respectively, in which one or more of the carbon atoms (and optionally any associated hydrogen atoms), are each, independently of one another, replaced with the same or different heteroatoms or heteroatomic groups. Typical heteroatoms or heteroatomic groups which can replace the carbon atoms include, but are not limited to, O, S, N, Si, —NH—, —S(O)—, —S(O)₂—, —S(O)NH—, —S(O)₂NH— and the like and combinations thereof. The heteroatoms or heteroatomic groups may be placed at any interior position of the alkyl, alkenyl or alkynyl groups. Examples of such heteroalkyl, heteroalkanyl, heteroalkenyl and/or heteroalkynyl groups include —CH₂—CH₂—O—CH₃, —CH₂—CH₂—NH—CH₃, —CH₂—CH₂—N(CH₃)—CH₃, —CH₂—S—CH₂, —CH₂—CH₂—S(O)—CH₃, —CH₂—CH₂—S(O)₂—CH₃, —CH═CH—O—CH₃, —CH₂—CH═N—O—CH₃, and —CH₂—CH₂—O—C═CH. For heteroalkyldiyl and heteroalkyleno groups, the heteratom or heteratomic group can also occupy either or both chain termini. For such groups, no orientation of the group is implied.

“Heteroaryl,” by itself or as part of another substituent, refers to a monovalent heteroaromatic radical derived by the removal of one hydrogen atom from a single atom of a parent heteroaromatic ring systems, as defined herein. Typical heteroaryl groups include, but are not limited to, groups derived from acridine, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. Preferably, the heteroaryl group comprises from 5 to 20 ring atoms (5-20 membered heteroaryl), more preferably from 5 to 10 ring atoms (5-10 membered heteroaryl). Preferred heteroaryl groups are those derived from furan, thiophene, pyrrole, benzothiophene, benzofuran, benzimidazole, indole, pyridine, pyrazole, quinoline, imidazole, oxazole, isoxazole and pyrazine.

“Heteroarylalkyl” by itself or as part of another substituent refers to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylakenyl and/or heteroarylalkynyl is used. In preferred embodiments, the heteroarylalkyl group is a 6-21 membered heteroarylalkyl, e.g., the alkanyl, alkenyl or alkynyl moiety of the heteroarylalkyl is (C1-C6) alkyl and the heteroaryl moiety is a 5-15-membered heteroaryl. In particularly preferred embodiments, the heteroarylalkyl is a 6-13 membered heteroarylalkyl, e.g., the alkanyl, alkenyl or alkynyl moiety is (C1-C3) alkyl and the heteroaryl moiety is a 5-10 membered heteroaryl.

“Parent Aromatic Ring System” refers to an unsaturated cyclic or polycyclic ring system having a conjugated π electron system. Specifically included within the definition of “parent aromatic ring system” are fused ring systems in which one or more of the rings are aromatic and one or more of the rings are saturated or unsaturated, such as, for example, fluorene, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Parent Heteroaromatic Ring System” refers to a parent aromatic ring system in which one or more carbon atoms (and optionally any associated hydrogen atoms) are each independently replaced with the same or different heteroatom. Typical heteroatoms to replace the carbon atoms include, but are not limited to, N, P, O, S, Si, etc. Specifically included within the definition of “parent heteroaromatic ring system” are fused ring systems in which one or more of the rings are aromatic and one or more of the rings are saturated or unsaturated, such as, for example, benzodioxan, benzofuran, chromane, chromene, indole, indoline, xanthene, etc. Typical parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Metal ion” or “Metal Salt” refers to a salt of a compound of the invention which is made with counterions understood in the art to be generally acceptable for pharmaceutical uses and which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine, morpholine, piperidine, dimethylamine, diethylamine and the like. Also included are salts of amino acids such as arginates and the like, and salts of organic acids like glucurmic or galactunoric acids and the like (see, e.g., Berge et al., 1977, J. Pharm. Sci. 66:1-19).

“Pharmaceutically acceptable vehicle” refers to a diluent, adjuvant, excipient or carrier with which a compound of the invention is administered.

“Substituted,” when used to modify a specified group or radical, means that one or more hydrogen atoms of the specified group or radical are each, independently of one another, replaced with the same or different substituent(s). Substituent groups useful for substituting saturated carbon atoms in the specified group or radical include, but are not limited to —R^{a}, halo, —O⁻, ═O, —OR^{b}, —SR^{b}, —S⁻, ═S, —NR^{c}R^{c}, ═NR^{b}, ═N—OR^{b}, trihalomethyl, —CF₃, —CN, —OCN, —SCN, —NO, —NO₂, ═N₂, —N₃, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a} is selected from the group consisting of alkyl, cycloalkyl, heteroalkyl, cycloheteroalkyl, aryl, arylalkyl, heteroaryl and heteroarylalkyl; each R^{b} is independently hydrogen or R^{a}; and each R^{c} is independently R^{b} or alternatively, the two R^{c}s are taken together with the nitrogen atom to which they are bonded form a 5-, 6- or 7-membered cycloheteroalkyl which may optionally include from 1 to 4 of the same or different additional heteroatoms selected from the group consisting of O, N and S. As specific examples, —NR^{c}R^{c} is meant to include —NH₂, —NH-alkyl, N-pyrrolidinyl and N-morpholinyl.

Similarly, substituent groups useful for substituting unsaturated carbon atoms in the specified group or radical include, but are not limited to, —R^{a}, halo, —O—, —OR, —SR^{b}, —S—, —NR^{c}R^{c}, trihalomethyl, —CF₃, —CN, —OCN, —SCN, —NO, —NO₂, —N₃, —S(O)₂R^{b}, —S(O)₂O⁻, 13 S(O)₂OR^{b}, —OS(O)₂R^{b}, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined.

Substituent groups useful for substituting nitrogen atoms in heteroalkyl and cycloheteroalkyl groups include, but are not limited to, —R^{a}, —O—, —OR^{b}, —SR^{b}, —S⁻, —NR^{c}R^{c}, trihalomethyl, —CF₃, —CN, —NO, —NO₂, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)OR^{b}, —OC(S)OR^{b}, —NR^{b}C(O)R^{b}, —NR^{b}C(S)R^{b}, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined.

Substituent groups from the above lists useful for substituting other specified groups or atoms will be apparent to those of skill in the art.

The substituents used to substitute a specified group can be further substituted, typically with one or more of the same or different groups selected from the various groups specified above.

“Sulfamoyl,” by itself or as part of another substituent, refers to a radical of the formula —S(O)₂NR′R″, where R′ and R″ are each, independently of one another, selected from the group consisting of hydrogen, alkyl and cycloalkyl as defined herein, or alternatively, R′ and R″, taken together with the nitrogen atom to which they are bonded, form a 5-, 6- or 7-membered cycloheteroalkyl ring as defined herein, which may optionally include from 1 to 4 of the same or different additional heteroatoms selected from the group consisting of O, S and N.

Methods of Synthesis

The particular phthaleins described above can be obtained via synthetic methods illustrated below. It should be understood that in R², R³, R⁵, R⁶, R⁷, R⁸, R⁹ and R¹⁰, are as previously defined for structural formula (I) and (Ia).

Starting materials useful for preparing compounds of the invention and intermediates thereof are commercially available or can be prepared by well-known synthetic methods (see, e.g., Harrison et al., “Compendium of Synthetic Organic Methods”, Vols. 1-8 (John Wiley and Sons, 1971-1996); “Beilstein Handbook of Organic Chemistry,” Beilstein Institute of Organic Chemistry, Frankfurt, Germany; Feiser et al., “Reagents for Organic Synthesis,” Volumes 1-21, Wiley Interscience; Trost et al., “Comprehensive Organic Synthesis,” Pergamon Press, 1991; “Theilheimer's Synthetic Methods of Organic Chemistry,” Volumes 1-45, Karger, March 1991; “Advanced Organic Chemistry,” Wiley Interscience, 1991; Larock “Comprehensive Organic Transformations,” VCH Publishers, 1989; Paquette, “Encyclopedia of Reagents for Organic Synthesis,” 3d Edition, John Wiley & Sons, 1995). Other methods for synthesis of the compounds described herein and/or starting materials are either described in the art or will be readily apparent to the skilled artisan.

A typical synthesis is depicted in Scheme I, wherein 2 equivalents of a phenol or phenol equivalent are condensed with 1 equivalent of a phthalic anhydride or equivalent under essentially acid -anhydrous conditions.

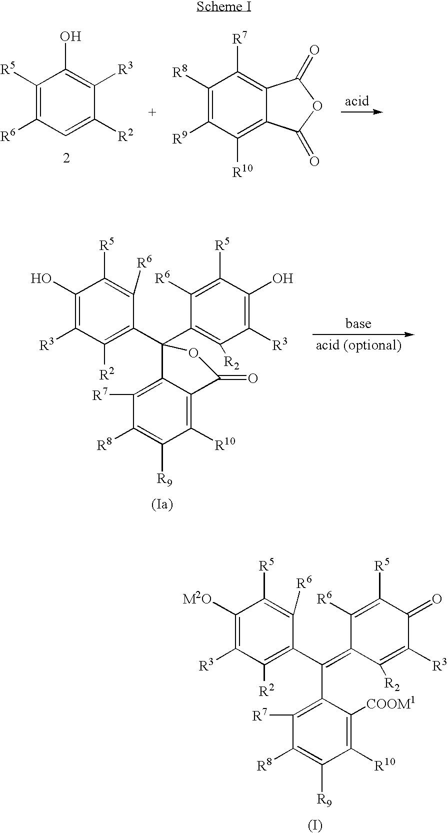

Generally, the phenol and anhydride are condensed in the presence of an acid under anhydrous conditions. For example, polyphosphoric acid and zinc chloride can be utilized. The carbon atom at 4-position -position with respect to the aromatic hydroxyl group must not be substituted as it is necessary for reaction. Polyphosphoric acid acts as a condensing agent as well as reaction medium. The reaction with only polyphosphoric acid afforded tarry products but when very small amount of zinc chloride was added to polyphosphoric acid, clean product was isolated. Very small amount of zinc chloride was found to increase yield and purity of the product. Polyphosphoric acid can be replaced with orthophosphoric acid, chlorosulfonic acid, methane sulfonic acid, trifluoroacetic acid or other acids under anhydrous conditions. Suitable solvents include non-protic solvents known in the art such as tetrahydrofuran, dioxane, methylene chloride, ether, etc.

The reaction proceeds with the formation of an isobenzofuranone (Ia), which is then treated with a base under aqueous conditions. The salt can be isolated or the solution can be acidified to produce the protonated phenol/carboxylic acid. For example, one molar equivalent of Ia was condensed with either two molar equivalent of sodium hydroxide in 85% ethanol or two molar equivalent of sodium ethoxide in ethanol. The products are generally solids and can be easily purified via filtration, crystallization, and other methods known in the art.

Suitable phenols include, but are not limited to 2-nitrophenol, 3-nitrophenol, 2-chlorophenol, 3-chlorophenol, 2-bromophenol, 3-bromophenol, 2-iodophenol, 3-iodophenol, 2-fluorophenol, 3-fluorophenol, 2-aminophenol, 3-aminophenol, 2-acetamidophenol, 3-acetamidophenol, 2-cyanophenol, 3-cyanophenol, 2-methylphenol, 3-methylphenol, 2-ethylphenol, 3-ethylphenol, 2-proylphenol, 3-proylphenol, 2-isoproylphenol, 3-isoproylphenol, 2-butylphenol, 3-butylphenol, 2-isobutylphenol, 3-isobutylphenol, 2-pentylphenol, 3-pentylphenol 2-hexylphenol, 3-hexylphenol, 2-heptylphenol, 3-heptylphenol, 2-octylphenol, 3-octylphenol, 2-nonylphenol, 3-nonylphenol, 2-decylphenol, 3-decylphenol, 2-decylphenol, 2-methoxyphenol, 3-methoxyphenol, 2-ethoxyphenol, 3-ethoxyphenol, 2-propoxyphenol, 3-propoxyphenol, 2-isopropoxyphenol, 3-isopropoxyphenol, 2-butoxyphenol, 3-butoxyphenol, 2-isobutoxyphenol, 3-isobutoxylphenol, 2-allylphenol, 3-allylphenol, 2-vinylphenol, 3-vinylphenol, 2-phenylphenol, 3-phenylphenol, 2-phenoxyphenol, 3-phenoxyphenol, 2-cyclopropylphenol, 3-cyclopropylphenol, 2-cyclobutylphenol, 3-cyclobutylphenol, 2-cyclopentylphenol, 3-cyclopentylphenol, 2-cyclohexylphenol, 3-cyclohexylphenol, 2-cycloheptylphenol, 3-cycloheptylphenol, 2-cyclooctylphenol, 3-cyclooctylphenol, 2-cyclononylphenol, 3-cyclononylphenol, 2-cyclodecylphenol, 3-cyclodecylphenol, 2,3-dinitrophenol, 2,5-dinitrophenol, 2,6-dinitrophenol, 2,3-dimethylphenol, 2,5-dimethylphenol, 2,6-dimethylphenol, 2,3-diethylphenol, 2,5-diethylphenol, 2,6-diethylphenol, 2,3-diproplylphenol, 2,5-dipropylphenol, 2,6-dipropylphenol, 2,3-diisoproplylphenol, 2,5-diisopropylphenol, 2,6-diisopropylphenol, 2,3-dibutylphenol, 2,5-dibutylphenol, 2,6-dibutylphenol, 2,3-diisobutylphenol, 2,5-diisobutylphenol, 2,6-diisobutylphenol, 2,3-dipentylphenol, 2,5-dipentylphenol, 2,6-dipentylphenol, 2,3-dihexylphenol, 2,5-dihexylphenol, 2,6-dihexylphenol, 2,3-diheptylphenol, 2,5-diheptylphenol, 2,6-diheptylphenol, 2,3-dioctylphenol, 2,5-dioctylphenol, 2,6-dioctylphenol, 2,3-dinonylphenol, 2,5-dinonylphenol, 2,6-dinonylphenol, 2,3-didecylphenol, 2,5-didecylphenol, 2,6-didecylphenol, 2,3-dimethoxyphenol, 2,5-dimethoxyphenol, 2,6-dimethoxyphenol, 2,3-diethoxyphenol, 2,5-diethoxyphenol, 2,6-diethoxyphenol, 2,3-dipropoxyphenol, 2,5-dipropoxyphenol, 2,6-dipropoxyphenol, 2,3-diisopropoxyphenol, 2,5-diisopropoxyphenol, 2,6-diisopropoxyphenol, 2,3-dibutoxyphenol, 2,5-dibutoxyphenol, 2,6-dibutoxyphenol, 2,3-diisobutoxyphenol, 2,5-diisobutoxyphenol, 2,6-diisobutoxyphenol, 2,3-dipentoxyphenol, 2,5-dipentoxyphenol, 2,6-dipentoxyphenol, 2,3-dihexoxyphenol, 2,5-dihexoxyphenol, 2,6-dihexoxyphenol, 2,3-diheptoxyphenol, 2,5-diheptoxyphenol, 2,6-diheptoxyphenol, 2,3-dioctoxyphenol, 2,5-dioctoxyphenol, 2,6-dioctoxyphenol, 2,3-dinonoxyphenol, 2,5-dinonoxyphenol, 2,6-dinonoxyphenol, 2,3-didecyloxyphenol, 2,5-didecyloxyphenol, 2,6-didecyloxyphenol, 2,3-dichlorophenol, 2,5-dichlorophenol, 2,6-dichlorophenol, 2,3-dibromophenol, 2,5-dibromophenol, 2,6-dibromophenol, 2,3-diiodophenol, 2,5-diiodophenol, 2,6-diiodophenol, 2,3-difluorophenol, 2,5-difluorophenol, 2,6-difluorophenol, 2,3-diaminophenol, 2,5-diaminophenol, 2,6-diaminophenol, 2,3-diacetamidophenol, 2,5-diacetamidophenol, 2,6-diacetamidophenol, 2,3-dicyanophenol, 2,5-dicyanophenol, 2,6-dicyanophenol, 2,3-diallylphenol, 2,5-diallylphenol, 2,6-diallylphenol, 2,3-divinylphenol, 2,5-divinylphenol, 2,6-divinylphenol, 2,3-diphenylphenol, 2,5-diphenylphenol, 2,6-diphenylphenol, 2,3-diphenoxyphenol, 2,5-diphenoxyphenol, 2,6-diphenoxyphenol, 2,3-dicycloproylphenol, 2,5-dicyclopropylphenol, 2,6-dicyclopropylphenol, 2,3-dicyclobutylphenol, 2,5-dicyclobutylphenol, 2,6-dicyclobutylphenol, 2,3-dicyclopentylphenol, 2,5-dicyclopentylphenol, 2,6-dicyclopentylphenol, 2,3-dicyclohexylphenol, 2,5-dicyclohexylphenol, 2,6-dicyclohexylphenol, 2,3-dicycloheptylphenol, 2,5-dicycloheptylphenol, 2,6-dicycloheptylphenol, 2,3-dicyclooctylphenol, 2,5-dicyclooctylphenol, 2,6-dicyclooctylphenol, 2,3-dicyclononylphenol, 2,5-dicyclononylphenol, 2,6-dicyclononylphenol, 2,3-dicyclodecylphenol, 2,5-dicyclodecylphenol, 2,6-dicyclodecylphenol, 2,3,5-trimethylphenol, 2,3,6-trimethylphenol 2,3,5-triethylphenol, 2,3,6-triethylphenol, 2,3,5-tripropylphenol, 2,3,6-tripropylphenol, 2,3,5-tributylphenol, 2,3,6-tributylphenol, 2,3,5-trichlorophenol, 2,3,6-trichlorophenol, 2,3,5-tribromophenol, 2,3,6-tribromophenol, 2,3,5-triiodophenol, 2,3,6-triiodophenol, 2,3,5-trifluorophenol, 2,3,6-trifluorophenol, 2,3,5-trivinylphenol, 2,3,6-trivinylphenol, 2,3,5-triallylphenol, 2,3,6-triallylphenol, 2,3,5-triphenylphenol, 2,3,6-triphenylphenol, 2,3,5-triphenoxyphenol, 2,3,6-triphenoxyphenol, 2,3,5-trimethoxyphenol, 2,3,6-trimethoxyphenol, 2,3,5-triethoxyphenol, 2,3,6-triethoxyphenol, 2,3,5-tripropoxyphenol, 2,3,6-tripropoxyphenol, 2,3,5-tributoxyphenol, 2,3,6-tributoxyphenol, 2,3,5-trinitrophenol, 2,3,6-trinitrophenol, 2,3,5-triaminophenol, 2,3,6-triaminophenol, 2,3,5-triacetamidophenol, 2,3,6-triacetamidophenol, 2,3,5-tricyanophenol, 2,3,6-tricyanophenol, 3-(N,N-diethylamino)phenol, 2-tert-butyl-5-methylphenol, 2-tert-butyl-6-methylphenol, 3-methyl-2-nitrophenol, 5-methyl-2-nitrophenol, 6-methyl-2-nitrophenol, 3-ethyl-2-nitrophenol, 5-ethyl-2-nitrophenol, 6-ethyl-2-nitrophenol, 3-methoxyl-2-nitrophenol, 5-methoxy-2-nitrophenol, 6-methoxy-2-nitrophenol, 1-naphthaol, 2-naphthaol, 2-nitro-1-naphthol, 3-nitro-1-naphthol, 5-nitro-1-naphthol, 6-nitro-1-naphthol, 7-nitro-1-naphthol, 8-nitro-1-naphthol, 2-methyl-1-naphthol, 3-methyl-1-naphthol, 5-methyl-1-naphthol, 6-methyl-1-naphthol, 7-methyl-1-naphthol, 8-methyl-1-naphthol, 2-methoxy-1-naphthol, 3-methoxy-1-naphthol, 5-methoxy-1-naphthol, 6-methoxy-1-naphthol, 7-methoxy-1-naphthol, 8-methoxy-1-naphthol, 2-chloro-1-naphthol, 3-chloro-1-naphthol, 5-chloro-1-naphthol, 6-chloro-1-naphthol, 7-chloro-1-naphthol, 8-chloro-1-naphthol, 2-bromo-1-naphthol, 3-bromo-1-naphthol, 5-bromo-1-naphthol, 6-bromo-1-naphthol, 7-bromo-1-naphthol, 8-bromo-1-naphthol, 2-iodo-1-naphthol, 3-iodo-1-naphthol, 5-iodo-1-naphthol, 6-iodo-1-naphthol, 7-iodo-1-naphthol, 8-iodo-1-naphthol, 2-fluoro-1-naphthol, 3-fluoro-1-naphthol, 5-fluoro-1-naphthol, 6-fluoro-1-naphthol, 7-bromo-1-naphthol, 8-fluoro-1-naphthol, 2-cyano-1-naphthol, 3-cyano-1-naphthol, 5-cyano-1-naphthol, 6-cyano-1-naphthol, 7-cyano-1-naphthol, 8-cyano-1-naphthol, 8-hydroxyquinaldine and 2-quinoxalinol.

The term “phenol equivalent” is intended to include those compounds where, as described above, R² and R³, for example, form an aromatic, heterocyclic, or non-aromatic ring. Suitable compounds include naphthols for example.

Suitable phthalic anhydrides include but are not limited to phthalic anhydride, 3-nitrophthalic anhydride, 4-nitrophthalic anhydride, 5-nitrophthalic anhydride, 6-nitrophthalic anhydride, 3-chlorophthalic anhydride, 4-chlorophthalic anhydride, 5-chlorophthalic anhydride, 6-chlorophthalic anhydride, 3-bromophthalic anhydride, 4-bromophthalic anhydride, 5-bromophthalic anhydride, 6-bromophthalic anhydride, 3-iodophthalic anhydride, 4-iodophthalic anhydride, 5-iodophthalic anhydride, 6-iodophthalic anhydride, 3-fluorophthalic anhydride, 4-fluorophthalic anhydride, 5-fluorophthalic anhydride, 6-fluorophthalic anhydride, 3-methylphthalic anhydride, 4-methylphthalic anhydride, 5-methylphthalic anhydride, 6-methylphthalic anhydride, 3-ethylphthalic anhydride, 4-ethylphthalic anhydride, 5-ethylphthalic anhydride, 6-ethylphthalic anhydride, 3-methoxyphthalic anhydride, 4-methoxyphthalic anhydride, 5-methoxyphthalic anhydride, 6-methoxyphthalic anhydride, 3-cyanophthalic anhydride, 4-cyanophthalic anhydride, 5-cyanophthalic anhydride, 6-cyanophthalic anhydride, 3-aminophthalic anhydride, 4-aminophthalic anhydride, 5-aminophthalic anhydride, 6-aminophthalic anhydride, 3-acetamidophthalic anhydride, 4-acetamidophthalic anhydride, 5-acetamidophthalic anhydride, 6-acetamidophthalic anhydride, 3,4,5,6-tetrachlorophthalic anhydride, 3,4,5,6-tetrabromophthalic anhydride, 3,4,5,6-tetraiodophthalic anhydride, 3,4,5,6-tetrafluorophthalic anhydride, 3,4,5,6-tetranitrophthalic anhydride, 3,4,5,6-tetramethylphthalic anhydride, 3,4,5,6-tetraethylphthalic anhydride, 3,4,5,6-tetramethoxyphthalic anhydride, 3,4,5,6-tetracyanophthalic anhydride, 3,4,5,6-tetraaminophthalic anhydride, 3,4,5,6-tetraacetamidophthalic anhydride, naphthalic anhydride, 2-chloronaphthalic anhydride, 3-chloronaphthalic anhydride, 4-chloronaphthalic anhydride, 5-chloronaphthalic anhydride, 6-chloronaphthalic anhydride, 7-chloronaphthalic anhydride, 2-bromonaphthalic anhydride, 3-bromonaphthalic anhydride, 4-bromonaphthalic anhydride, 5-bromonaphthalic anhydride, 6-bromonaphthalic anhydride, 7-bromonaphthalic anhydride, 2-iodonaphthalic anhydride, 3-iodonaphthalic anhydride, 4-iodonaphthalic anhydride, 5-iodonaphthalic anhydride, 6-iodonaphthalic anhydride, 7-iodonaphthalic anhydride, 2-fluoronaphthalic anhydride, 3-fluoronaphthalic anhydride, 4-fluoronaphthalic anhydride, 5-fluoronaphthalic anhydride, 6-fluoronaphthalic anhydride, 7-fluoronaphthalic anhydride, 2-nitronaphthalic anhydride, 3-nitronaphthalic anhydride, 4-nitronaphthalic anhydride, 5-nitronaphthalic anhydride, 6-nitronaphthalic anhydride and 7-nitronaphthalic anhydride.

The term “phthalic anhydride equivalent” is intended to include those compounds where, as described above, R⁷ and R⁸, for example, form an aromatic, heterocyclic, or non-aromatic ring. Suitable compounds include naphthols for example.

Synthesis of Phenols and Hydrazides

The compounds of the invention may be obtained via synthetic methods illustrated below. It should be understood that in R², R³, R⁴, R⁵, R⁶, R⁷, R⁸, R⁹, R¹⁰, R¹¹, R¹², R¹³, R¹⁴ and R¹⁵ are as previously defined for structural formulae (II), (III), (IIIa) and (IV).

Starting materials useful for preparing compounds of the invention and intermediates thereof are commercially available or can be prepared by well-known synthetic methods (see, e.g., Harrison et al., “Compendium of Synthetic Organic Methods”, Vols. 1-8 (John Wiley and Sons, 1971-1996); “Beilstein Handbook of Organic Chemistry,” Beilstein Institute of Organic Chemistry, Frankfurt, Germany; Feiser et al., “Reagents for Organic Synthesis,” Volumes 1-21, Wiley Interscience; Trost et al., “Comprehensive Organic Synthesis,” Pergamon Press, 1991; “Theilheimer's Synthetic Methods of Organic Chemistry,” Volumes 1-45, Karger, March 1991; “Advanced Organic Chemistry,” Wiley Interscience, 1991; Larock “Comprehensive Organic Transformations,” VCH Publishers, 1989; Paquette, “Encyclopedia of Reagents for Organic Synthesis,” 3d Edition, John Wiley & Sons, 1995). Other methods for synthesis of the compounds described herein and/or starting materials are either described in the art or will be readily apparent to the skilled artisan.

A typical synthesis for substituted phenols is depicted in Scheme II, wherein a phenol is treated with a base to form the phenolic salt. Advantageously, the phenolic salts are water soluble, which is useful in the applications detailed throughout the specification.

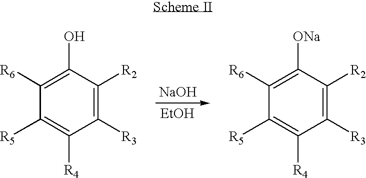

Generally, the phenol mixed with the base and the salt is formed. The solution may be heated to facilitate the rate of reaction.

Suitable phenols include, but are not limited to 2-nitrophenol, 3-nitrophenol, 4-nitrophenol, 2-chlorophenol, 3-chlorophenol, 4-chlorophenol, 2-bromophenol, 3-bromophenol, 4-bromophenol, 2-iodophenol, 3-iodophenol, 4-iodophenol, 2-aminophenol, 3-aminophenol, 4-aminophenol, 2-cyanophenol, 3-cyanophenol, 4-cyanophenol, 2-vinylphenol, 3-vinylphenol, 4-vinylphenol, 2,3-dichlorophenol, 2,4-dichlorophenol, 2,5-dichlorophenol, 2,6-dichlorophenol, 2,3-dibromophenol, 2,4-dibromophenol, 2,5-dibromophenol, 2,6-dibromophenol, 2,3-diiodophenol, 2,4-diiodophenol, 2,5-diiodophenol, 2,6-diiodophenol, 2,3-diaminophenol, 2,4-diaminophenol, 2,5-diaminophenol, 2,6-diaminophenol, 2,3-dicyanophenol, 2,4-dicyanophenol, 2,5-dicyanophenol, 2,6-dicyanophenol, 2,3-divinylphenol, 2,4-divinylphenol, 2,5-divinylphenol, 2,6-divinylphenol, 2,3-diphenylphenol, 2,3,4-trichlorophenol, 2,3,5-trichlorophenol, 2,3,6-trichlorophenol, 2,3,4-tribromophenol, 2,3,5-tribromophenol, 2,3,6-tribromophenol, 2,3,4-triiodophenol, 2,3,5-triiodophenol, 2,3,6-triiodophenol, 2,3,4-trivinylphenol, 2,3,5-trivinylphenol, 2,3,6-trivinylphenol, 2,3,4-trinitrophenol, 2,3,5-trinitrophenol, 2,3,6-trinitrophenol, 2,3,4-triaminophenol, 2,3,5-triaminophenol, 2,3,6-triaminophenol, 2,3,4-tricyanophenol, 2,3,5-tricyanophenol, 2,3,6-tricyanophenol, 3-(N,N-diethylamino)phenol, 3-methyl-2-nitrophenol, 5-methyl-2-nitrophenol, 6-methyl-2-nitrophenol, 3-ethyl-2-nitrophenol, 5-ethyl-2-nitrophenol, 6-ethyl-2-nitrophenol, 3-methoxyl-2-nitrophenol, 5-methoxy-2-nitrophenol, 6-methoxy-2-nitrophenol, 2-nitro-1-naphthol, 3-nitro-1-naphthol, 4-nitro-1-naphthol, 5-nitro-1-naphthol, 6-nitro-1-naphthol, 7-nitro-1-naphthol, 8-nitro-1-naphthol, 2-chloro-1-naphthol, 3-chloro-1-naphthol, 4-chloro-1-naphthol, 5-chloro-1-naphthol, 6-chloro-1-naphthol, 7-chloro-1-naphthol, 8-chloro-1-naphthol, 2-bromo-1-naphthol, 3-bromo-1-naphthol, 4-bromo-1-naphthol, 5-bromo-1-naphthol, 6-bromo-1-naphthol, 7-bromo-1-naphthol, 8-bromo-1-naphthol, 2-iodo-1-naphthol, 3-iodo-1-naphthol, 4-iodo-1-naphthol, 5-iodo-1-naphthol, 6-iodo-1-naphthol, 7-iodo-1-naphthol, 8-iodo-1-naphthol, 2-cyano-1-naphthol, 3-cyano-1-naphthol, 4-cyano-1-naphthol, 5-cyano-1-naphthol, 6-cyano-1-naphthol, 7-cyano-1-naphthol, 8-cyano-1-naphthol and 8-hydroxyquinaldine.

The term “phenol equivalent” is intended to include those compounds where, as described above, R² and R³, for example, form an aromatic, heterocyclic, or non-aromatic ring. Suitable compounds include naphthols for example.

A typical synthesis of hydrazines is depicted in Scheme III, where a hydrazine (NH₂NH—R¹⁵, wherein R¹⁵ can be a hydrogen atom or as described above) and an ester are condensed to form the hydrazide.

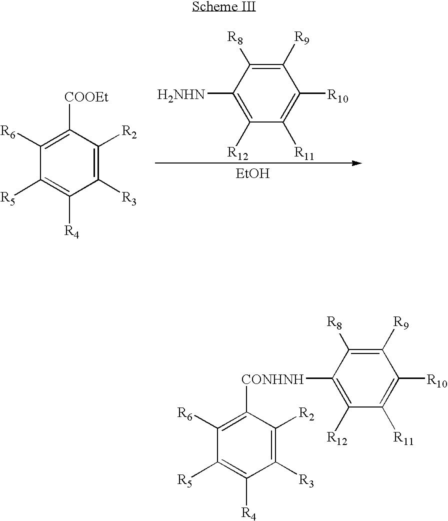

Typically the ester and the hydrazine are combined in a solvent, such as a protic solvent, e.g., an alcohol, such as ethanol, and heated, e.g., to reflux. Upon cooling, the hydrazide generally precipitates from solution and can be collected.

Suitable salicylic derivatives include, but not limited to salicylic acid, 3-methylsalicylic acid, 4-methylsalicylic acid, 5-methylsalicylic acid, 6-methylsalicylic acid, 3-ethylsalicylic acid, 4-ethylsalicylic acid, 5-ethylsalicylic acid, 6-ethylsalicylic acid, 3-propylsalicylic acid, 4-propylsalicylic acid, 5-propylsalicylic acid, 6-propylsalicylic acid, 3-isopropylsalicylic acid, 4-isopropylsalicylic acid, 5-isopropylsalicylic acid, 6-isopropylsalicylic acid, 3-butylsalicylic acid, 4-butylsalicylic acid, 5-butylsalicylic acid, 6-butylsalicylic acid, 3-isobutylsalicylic acid, 4-isobutylsalicylic acid, 5-isobutylsalicylic acid, 6-isobutylsalicylic acid, 3-methoxysalicylic acid, 4-methoxysalicylic acid, 5-methoxysalicylic acid, 6-methoxysalicylic acid, 3-ethoxysalicylic acid, 4-ethoxysalicylic acid, 5-ethoxysalicylic acid, 6-ethoxysalicylic acid, 3-propoxysalicylic acid, 4-propoxysalicylic acid, 5-propoxysalicylic acid, 6-propoxysalicylic acid, 3-butoxysalicylic acid, 4-butoxysalicylic acid, 5-butoxysalicylic acid, 6-butoxysalicylic acid, 3-nitrosalicylic acid, 4-nitrosalicylic acid, 5-nitrosalicylic acid, 6-nitrosalicylic acid, 3-chlorosalicylic acid, 4-chlorosalicylic acid, 5-chlorosalicylic acid, 6-chlorosalicylic acid, 3-bromosalicylic acid, 4-bromosalicylic acid, 5-bromosalicylic acid, 6-bromosalicylic acid, 3-iodosalicylic acid, 4-iodosalicylic acid, 5-iodosalicylic acid, 6-iodoosalicylic acid, 3-fluorosalicylic acid, 4-fluorosalicylic acid, 5-fluorosalicylic acid, 6-fluorosalicylic acid, 3-aminosalicylic acid, 4-aminosalicylic acid, 5-aminosalicylic acid, 6-aminosalicylic acid, 3-acetamidosalicylic acid, 4-acetamidosalicylic acid, 5-acetamidosalicylic acid, 6-acetamidosalicylic acid, 3-cyanosalicylic acid, 4-cyanosalicylic acid, 5-cyanosalicylic acid, 6-cyanosalicylic acid, 3-sulfosalicylic acid, 4-sulfosalicylic acid, 5-sulfosalicylic acid, 6-sulfosalicylic acid, 3,5-dimethylsalicylic acid, 3,5-diethylsalicylic acid, 3,5-dipropylsalicylic acid, 3,5-dibutylsalicylic acid, 3,5-dimethoxysalicylic acid, 3,5-diethoxysalicylic acid, 3,5-dipropoxysalicylic acid, 3,5-dibutoxysalicylic acid, 3,5-dichlorosalicylic acid, 3,5-dibromosalicylic acid, 3,5-diiodosalicylic acid, 3,5-difluorosalicylic acid, 3,5-dinitrosalicylic acid, 3,5-diaminosalicylic acid, 3,5-diacetamidosalicylic acid, 3,5-dicyanosalicylic acid, 3,5-disulfosalicylic acid, substituted/unsubstituted alkyl salicylic acid, substituted/unsubstituted alkoxy salicylic acid, substituted/unsubstituted aryl salicylic acid, substituted/unsubstituted cycloalkyl salicylic acid and substituted/unsubstituted hetaryl salicylic acid.

Suitable hydrazines include but not limited to hydrazine hydrate, 4-nitrophenylhydrazine, 3-nitrophenylhydrazine, 2-nitrophenylhydrazine, 4-nitrobenzoic hydrazide, 3-nitrobenzoic hydrazide, 2-nitrobenzoic hydrazide, p-toluenesulfonylhydrazide, m-toluenesulfonylhydrazide, o-toluenesulfonyl-hydrazide, 2,4-dinitrophenylhydrazine (2,4-DNP), 1-naphthoic hydrazide, 2-naphthoic hydrazide, nicotinic hydrazide, substituted/unsubstituted alkyl hydrazide, substituted/unsubstituted alkoxy hydrazide, substituted/unsubstituted aryl hydrazide, substituted/unsubstituted cycloalkyl hydrazide and substituted/unsubstituted hetaryl hydrazide.

In compositions where color is not desired (clear to color), a base is not included in the composition, but is provided, for example) by the surface of the substrate acted upon, i.e., the oral cavity, saliva, etc.

Alternatively, the composition can be basic and highly colored by use of a fugative base or a base that is not fugitive in nature, such as a metal hydroxide. The pH of the substrate will then determine whether the color of the composition is unchanged upon application, disappears or changes color. Therefore, by choice of dye and pH of the composition and pH of the surface of the substrate, compositions are provided that can be colored and remain so, can change from color to clear, or color to color, or uncolored to a color. It is the combination of the acid-base dye and the substrate surface that determines how the color change, or maintenance, is effected.

In general, the amount of color components incorporated into the oral care composition of the invention may range from approximately 0.01% to approximately 10% by weight.

In general, the oral care compositions of the invention contain no foaming agent since any significant amount of such an agent tends to diminish the visibility of the color change occurring and thereby render the color change less evident or perceptible. If a foaming agent incorporated in the oral care composition of the invention, no more than approximately 0.05% by weight should be present in order to minimize the disadvantageous effect imparted by foaming agents. Various conventional foaming agents may be used such as sodium lauryl sulfate, sodium N-lauroyl sarcosinate or cocomonoglyceride sulfonate may be used in the practice of the invention with the use of sodium lauryl sulfate being preferred.

The toothpaste of the invention may also contain other conventional ingredients or components such as gelling agents or binders, polishing agents, vehicles, humectants, flavoring agents, sweeteners, and a fluoride containing compound. Thus, from approximately 0.5 to approximately 18% by weight of a gelling agent or binder may be used, the gelling agent being selected from known gelling agents such as sodium carboxymethyl cellulose, xanthan gum, polyvinyl pyrrolidone, hydroxyethyl cellulose, polyvinyl alcohol, Irish moss extract, sodium alginate and mixtures thereof. From approximately 15% to approximately 90% by weight of a polishing agent such as silica gel, silica, sodium aluminum silicate, hydrated alumina, dicalcium phosphate, calcium pyrophosphate, calcium carbonate and mixtures thereof may be incorporated into the toothpaste of the invention. From approximately 10% to approximately 80% by weight of a humectant such as sorbitol, maltitol, polyethylene glycol, glycerin and mixtures thereof may be utilized in the practice of the invention. It is highly preferred that the toothpaste of the invention contain between approximately 0.1 and approximately 2% by weight of a fluoride with sodium fluoride or stannous fluoride being especially preferred. The flavoring agents and sweeteners known to those in the toothpaste art may constitute from approximately 0.1 to approximately 10% by weight of the toothpaste, and water constitutes the most common toothpaste vehicle.

In an optional embodiment of the invention, the toothpaste may contain a flavoring agent or component constituted by encapsulated or agglomerated flavoring crystals, ingredients or components known to those in the art and which upon mechanical agitation bring about a time release (5 to 20 second delayed) flavoring of the toothpaste. The incorporation of such a time released flavor burst in conjunction with the color changing properties of the toothpaste of the invention advantageously induces children to brush for an increased amount of time while encouraging them to utilize enough mechanical scrubbing force to properly brush their teeth.

As used herein, the term “toothpaste” is intended to encompass formulations of both the paste and gel form, the two forms being generally identical except that the paste form contains titanium dioxide. The components or ingredients discussed and enumerated above may be used in both the paste and gel forms of the present invention and, as previously indicated, a white paste containing titanium dioxide may be used as a partition interposed between separate layers of toothpaste containing the respective color components.

In an optional embodiment of the invention, the mouthwash may contain a flavoring agent or component, such as spearmint or wintergreen, or constituted by encapsulated or agglomerated flavoring crystals, ingredients or components known to those in the art and which upon mechanical agitation bring about a time release (5 to 20 second delayed) flavoring of the mouthwash. The incorporation of such a time released flavor burst in conjunction with the color changing properties of the mouthwash of the invention advantageously induces children to treat the oral cavity for an increased amount of time while encouraging them to utilize enough swishing force to properly treat their throat, oral cavity and teeth.

As used herein, the term “mouthwash” is intended to encompass aqueous based compositions that are known in the art and generally include water, a surfactant, a flavoring agent, optionally fluoride and optionally, an astringent, such as an alcohol. Mouthwashes, including plaque removing liquids, typically comprise a water/alcohol solution, flavour, humectant, sweetener, foaming agent, colorant, and optionally enzymes.

Suitable humectants for use in oral care products according to the invention include the following compounds and mixtures thereof: glycerol, polyol, sorbitol, polyethylene glycols (PEG), propylene glycol, 1,3-propanediol, 1,4-butanediol, hydrogenated partially hydrolysed polysaccharides and the like. Humectants are in general present in from 0% to 80% by weight in the mouthwash.

Suitable surfactant include, anionic, cationic, non-ionic, amphoteric and/or zwitterionic surfactants. These can be present at levels of from about 0.01% to about 15%, particularly from about 0.1 to about 13%, more particularly from about 0.25 to about 10% by weight of the final product.

Examples of anionic, nonionic, cationic and amphoteric surface-active agents that are suitable for use in the present invention are described in Kirk-Othmer, Encyclopedia of Chemical Technology, Third Edition, Volume 22, pages 347-387, and McCutcheon's Detergents and Emulsifiers, North American Edition, 1983, both of which are incorporated herein by reference.

Surfactants, for example, include fatty alcohol sulphates, salts of sulphonated mono-glycerides or fatty acids having 10 to 20 carbon atoms, fatty acid-albumen condensation products, salts of fatty acids amides and taurines and/or salts of fatty acid esters of isethionic acid.

Suitable sweeteners include saccharin.

Flavours, such as spearmint, wintergreen and the like, are usually present in low amounts, such as from about 0.01% to about 5% by weight, especially from about 0. 1% to about 5%.

The following examples illustrate the practice of the invention.

A mouthwash of the invention (in weight % of the final mouthwash composition) can include the following ingredients:

| | |
|---|---|
| 0-20% | Humectant |
| 0-8% | Surfactant |
| 0-5% | disinfecting cleaners, antimicrobial agents, sweetners, |
| | flavors, preservatives, etc |
| 0-20% | Ethanol (or other pharmaceutically acceptable alcohol) |
| 0.01-5% | acid-base indicator of the invention |
| 0-90% | Water |

### MOUTHWASH EXAMPLES

### Example 1

| Chemical Component | Weight in grams |
|---|---|
| Pluronic F108 | 0.5 |
| Colonial SLS | 1 |
| Glycerin | 8 |
| Sodium saccharin | 0.05 |
| Zinc chloride | 0.005 |
| Methyl salicylate | 0.1 |
| Menthol | 0.1 |
| Thymolphthalein | 0.5 |
| Sodium hydroxide | 0.09 |
| Deionized water | 89.555 |
| Liquid Germall Plus | 0.1 |

In a beaker, deionized water (50 g) was stirred and heated at 50° C. Pluronic F108 was slowly added and reaction mixture was further stirred at 50° C. for 15 minutes. The mixture was cooled to room temperature followed by addition of Colonial SLS, glycerin, sodium saccharin, zinc chloride, methyl salicylate, menthol, thymolphthalein, sodium hydroxide, liquid germall plus and remaining deionized water. The reaction mixture was further stirred for 2 hours at room temperature.
Color of the solution: Blue
Color change: Blue to colorless

### Example 2

| Chemical Component | Weight in grams |
|---|---|
| Pluronic F108 | 0.5 |
| Colonial SLS | 1 |
| Glycerin | 8 |
| Sodium saccharin | 0.05 |
| Zinc chloride | 0.005 |
| Methyl salicylate | 0.1 |
| Menthol | 0.1 |
| o-Cresolphthalein | 0.5 |
| Sodium hydroxide | 0.11 |
| Deionized water | 89.535 |
| Liquid Germall Plus | 0.1 |

In a beaker, deionized water (50 g) was stirred and heated at 50° C. Pluronic F108 was slowly added and reaction mixture was further stirred at 50° C. for 15 minutes. The mixture was cooled to room temperature followed by addition of Colonial SLS, glycerin, sodium saccharin, zinc chloride, methyl salicylate, menthol, o-cresolphthalein, sodium hydroxide, liquid germall plus and remaining deionized water. The reaction mixture was further stirred for 2 hours at room temperature.
Color of the solution: Magenta
Color change: Magenta to colorless

### Example 3

| Chemical Component | Weight in grams |
|---|---|
| Pluronic F108 | 0.5 |
| Colonial SLS | 1 |
| Glycerin | 8 |
| Sodium saccharin | 0.05 |
| Zinc chloride | 0.005 |
| Methyl salicylate | 0.1 |
| Menthol | 0.1 |
| 3,3-bis-(4-hydroxy-3-phenylphenyl)- | 0.5 |
| 1-(3H)-isobenzofuranone | |
| Sodium hydroxide | 0.08 |
| Deionized water | 89.565 |
| Liquid Germall Plus | 0.1 |

In a beaker, deionized water (50 g) was stirred and heated at 50° C. Pluronic F108 was slowly added and reaction mixture was further stirred at 50° C. for 15 minutes. The mixture was cooled to room temperature followed by addition of Colonial SLS, glycerin, sodium saccharin, zinc chloride, methyl salicylate, menthol, 3,3-bis-(4-hydroxy-3-phenylphenyl)-1-(3H)-isobenzofuranone, sodium hydroxide, liquid germall plus and remaining deionized water. The reaction mixture was further stirred for 2 hours at room temperature.
Color of the solution: Purple
Color change: Purple to colorless

### Example 4

| Chemical Component | Weight in grams |
|---|---|
| Pluronic F108 | 0.5 |
| Colonial SLS | 1 |
| Glycerin | 8 |
| Sodium saccharin | 0.05 |
| Zinc chloride | 0.005 |
| Methyl salicylate | 0.1 |
| Menthol | 0.1 |
| 3,3-bis-(4-hydroxy-3,5-diisopropylphenyl)- | 0.5 |
| 1-(3H)-isobenzofuranone | |
| Sodium hydroxide | 0.08 |
| Deionized water | 89.565 |
| Liquid Germall Plus | 0.1 |

In a beaker, deionized water (50 g) was stirred and heated at 50° C. Pluronic F108 was slowly added and reaction mixture was further stirred at 50° C. for 15 minutes. The mixture was cooled to room temperature followed by addition of Colonial SLS, glycerin, sodium saccharin, zinc chloride, methyl salicylate, menthol, 3,3-bis-(4-hydroxy-3,5-diisopropylphenyl)-1-(3H)-isobenzofuranone, sodium hydroxide, liquid germall plus and remaining deionized water. The reaction mixture was further stirred for 2 hours at room temperature.
Color of the solution: Violet
Color change: Violet to colorless

### Example 5

| Chemical Component | Weight in grams |
|---|---|
| Pluronic F108 | 0.5 |
| Colonial SLS | 1 |
| Glycerin | 8 |
| Sodium saccharin | 0.05 |
| Zinc chloride | 0.005 |
| Methyl salicylate | 0.1 |
| Menthol | 0.1 |
| 3,3-bis-(4-hydroxy-3,5-dimethoxyphenyl)- | 0.5 |
| 1-(3H)-isobenzofuranone | |
| Sodium hydroxide | 0.09 |
| Deionized water | 89.555 |
| Liquid Germall Plus | 0.1 |

In a beaker, deionized water (50 g) was stirred and heated at 50° C. Pluronic F108 was slowly added and reaction mixture was further stirred at 50° C. for 15 minutes. The mixture was cooled to room temperature followed by addition of Colonial SLS, glycerin, sodium saccharin, zinc chloride, methyl salicylate, menthol, 3,3-bis-(4-hydroxy-3,5-dimethoxyphenyl)-1-(3H)-isobenzofuranone, sodium hydroxide, liquid germall plus and remaining deionized water. The reaction mixture was further stirred for 2 hours at room temperature.
Color of the solution: Teal
Color change: Teal to colorless

### Example 6

| Chemical Component | Weight in grams |
|---|---|
| Pluronic F108 | 0.5 |
| Colonial SLS | 1 |
| Glycerin | 8 |
| Sodium saccharin | 0.05 |
| Zinc chloride | 0.005 |
| Methyl salicylate | 0.1 |
| Menthol | 0.1 |
| 3,3-bis-(4-hydroxy-3,5-dimethylphenyl)- | 0.5 |
| 1-(3H)-isobenzofuranone | |
| Sodium hydroxide | 0.1 |
| Deionized water | 89.545 |
| Liquid Germall Plus | 0.1 |

In a beaker, deionized water (50 g) was stirred and heated at 50° C. Pluronic F108 was slowly added and reaction mixture was further stirred at 50° C. for 15 minutes. The mixture was cooled to room temperature followed by addition of Colonial SLS, glycerin, sodium saccharin, zinc chloride, methyl salicylate, menthol, 3,3-bis-(4-hydroxy-3,5-dimethylphenyl)-1-(3H)-isobenzofuranone, sodium hydroxide, liquid germall plus and remaining deionized water. The reaction mixture was further stirred for 2 hours at room temperature.
Color of the solution: Purple
Color change: Purple to colorless

### Example 7

| Chemical Component | Weight in grams |
|---|---|
| Pluronic F108 | 0.5 |
| Colonial SLS | 1 |
| Glycerin | 8 |
| Sodium saccharin | 0.05 |
| Zinc chloride | 0.005 |
| Methyl salicylate | 0.1 |
| Menthol | 0.1 |
| 3,3-bis-(4-hydroxy-3,6-diimethylphenyl)- | 0.5 |
| 1-(3H)-isobenzofuranone | |
| Sodium hydroxide | 0.1 |
| Deionized water | 89.545 |
| Liquid Germall Plus | 0.1 |

In a beaker, deionized water (50 g) was stirred and heated at 50° C. Pluronic F108 was slowly added and reaction mixture was further stirred at 50° C. for 15 minutes. The mixture was cooled to room temperature followed by addition of Colonial SLS, glycerin, sodium saccharin, zinc chloride, methyl salicylate, menthol, 3,3-bis-(4-hydroxy-3,6-diimethylphenyl)-1-(3H)-isobenzofuranone, sodium hydroxide, liquid germall plus and remaining deionized water. The reaction mixture was further stirred for 2 hours at room temperature.
Color of the solution: Blue
Color change: Blue to colorless

### Example 8

| Chemical Component | Weight in grams |
|---|---|
| Pluronic F108 | 0.5 |
| Colonial SLS | 1 |
| Glycerin | 8 |
| Sodium saccharin | 0.05 |
| Zinc chloride | 0.005 |
| Methyl salicylate | 0.1 |
| Menthol | 0.1 |
| 3,3-bis-(4-hydroxy-3-ethylphenyl)- | 0.5 |
| 1-(3H)-isobenzofuranone | |
| Sodium hydroxide | 0.1 |
| Deionized water | 89.545 |
| Liquid Germall Plus | 0.1 |

In a beaker, deionized water (50 g) was stirred and heated at 50° C. Pluronic F108 was slowly added and reaction mixture was further stirred at 50° C. for 15 minutes. The mixture was cooled to room temperature followed by addition of Colonial SLS, glycerin, sodium saccharin, zinc chloride, methyl salicylate, menthol, 3,3-bis-(4-hydroxy-3-ethylphenyl)-1-(3H)-isobenzofuranone, sodium hydroxide, liquid germall plus and remaining deionized water. The reaction mixture was further stirred for 2 hours at room temperature.
Color of the solution: Magenta
Color change: Magenta to colorless

### Example 9

| Chemical Component | Weight in grams |
|---|---|
| Pluronic F108 | 0.5 |
| Colonial SLS | 1 |
| Glycerin | 8 |
| Sodium saccharin | 0.05 |
| Zinc chloride | 0.005 |
| Methyl salicylate | 0.1 |
| Menthol | 0.1 |
| 3,3-bis-(4-hydroxy-3-isopropylphenyl)- | 0.5 |
| 1-(3H)-isobenzofuranone | |
| Sodium hydroxide | 0.09 |
| Deionized water | 89.555 |
| Liquid Germall Plus | 0.1 |

In a beaker, deionized water (50 g) was stirred and heated at 50° C. Pluronic F108 was slowly added and reaction mixture was further stirred at 50° C. for 15 minutes. The mixture was cooled to room temperature followed by addition of Colonial SLS, glycerin, sodium saccharin, zinc chloride, methyl salicylate, menthol, 3,3-bis-(4-hydroxy-3-isopropylphenyl)-1-(3H)-isobenzofuranone, sodium hydroxide, liquid germall plus and remaining deionized water. The reaction mixture was further stirred for 2 hours at room temperature.
Color of the solution: Pink
Color change: Pink to colorless

### Example 10

| Chemical Component | Weight in grams |
|---|---|
| Pluronic F108 | 0.5 |
| Colonial SLS | 1 |
| Glycerin | 8 |
| Sodium saccharin | 0.05 |
| Zinc chloride | 0.005 |
| Methyl salicylate | 0.1 |
| Menthol | 0.1 |
| 3,3-bis-(4-hydroxy-3-methoxyphenyl)- | 0.5 |
| 1-(3H)-isobenzofuranone | |
| Sodium hydroxide | 0.1 |
| Deionized water | 89.545 |
| Liquid Germall Plus | 0.1 |

In a beaker, deionized water (50 g) was stirred and heated at 50° C. Pluronic F108 was slowly added and reaction mixture was further stirred at 50° C. for 15 minutes. The mixture was cooled to room temperature followed by addition of Colonial SLS, glycerin, sodium saccharin, zinc chloride, methyl salicylate, menthol, 3,3-bis-(4-hydroxy-3-methoxyphenyl)-1-(3H)-isobenzofuranone, sodium hydroxide, liquid germall plus and remaining deionized water. The reaction mixture was further stirred for 2 hours at room temperature.
Color of the solution: Blue
Color change: Blue to colorless

### Example 11

| Chemical Component | Weight in grams |
|---|---|
| Pluronic F108 | 0.5 |
| Colonial SLS | 1 |
| Glycerin | 8 |
| Sodium saccharin | 0.05 |
| Zinc chloride | 0.005 |
| Methyl salicylate | 0.1 |
| Menthol | 0.1 |
| 3,3-bis-(4-hydroxy-2,3,5-trimethylphenyl)- | 0.5 |
| 1-(3H)-isobenzofuranone | |
| Sodium hydroxide | 0.09 |
| Deionized water | 89.555 |
| Liquid Germall Plus | 0.1 |

In a beaker, deionized water (50 g) was stirred and heated at 50° C. Pluronic F108 was slowly added and reaction mixture was further stirred at 50° C. for 15 minutes. The mixture was cooled to room temperature followed by addition of Colonial SLS, glycerin, sodium saccharin, zinc chloride, methyl salicylate, menthol, 3,3-bis-(4-hydroxy-2,3,5-trimethylphenyl)-1-(3H)-isobenzofuranone, sodium hydroxide, liquid germall plus and remaining deionized water. The reaction mixture was further stirred for 2 hours at room temperature.
Color of the solution: Teal
Color change: Teal to colorless

### Example 12

| Chemical Component | Weight in grams |
|---|---|
| Pluronic F108 | 0.5 |
| Colonial SLS | 1 |
| Glycerin | 8 |
| Sodium saccharin | 0.05 |
| Zinc chloride | 0.005 |
| Methyl salicylate | 0.1 |
| Menthol | 0.1 |
| Bromo-thymolphthalein | 0.5 |
| Sodium hydroxide | 0.08 |
| Deionized water | 89.565 |
| Liquid Germall Plus | 0.1 |

In a beaker, deionized water (50 g) was stirred and heated at 50° C. Pluronic F108 was slowly added and reaction mixture was further stirred at 50° C. for 15 minutes. The mixture was cooled to room temperature followed by addition of Colonial SLS, glycerin, sodium saccharin, zinc chloride, methyl salicylate, menthol, bromo-thymolphthalein, sodium hydroxide, liquid germall plus and remaining deionized water. The reaction mixture was further stirred for 2 hours at room temperature.
Color of the solution: Teal
Color change: Teal to colorless

### Example 13

| Chemical Component | Weight in grams |
|---|---|
| Pluronic F108 | 0.5 |
| Colonial SLS | 1 |
| Glycerin | 8 |
| Sodium saccharin | 0.05 |
| Zinc chloride | 0.005 |
| Methyl salicylate | 0.1 |
| Menthol | 0.1 |
| Bromo-o-cresolphthalein | 0.5 |
| Sodium hydroxide | 0.1 |
| Deionized water | 89.545 |
| Liquid Germall Plus | 0.1 |

In a beaker, deionized water (50 g) was stirred and heated at 50° C. Pluronic F108 was slowly added and reaction mixture was further stirred at 50° C. for 15 minutes. The mixture was cooled to room temperature followed by addition of Colonial SLS, glycerin, sodium saccharin, zinc chloride, methyl salicylate, menthol, bromo-o-cresolphthalein, sodium hydroxide, liquid germall plus and remaining deionized water. The reaction mixture was further stirred for 2 hours at room temperature.
Color of the solution: Purple
Color change: Purple to colorless

### Example 14

| Chemical Component | Weight in grams |
|---|---|
| Pluronic F108 | 0.5 |
| Colonial SLS | 1 |
| Glycerin | 8 |
| Sodium saccharin | 0.05 |
| Zinc chloride | 0.005 |
| Methyl salicylate | 0.1 |
| Menthol | 0.1 |
| 3,3-bis-(4-hydroxy-3-sec-butylphenyl)- | 0.5 |
| 1-(3H)-isobenzofuranone | |
| Sodium hydroxide | 0.08 |
| Deionized water | 89.565 |
| Liquid Germall Plus | 0.1 |

In a beaker, deionized water (50 g) was stirred and heated at 50° C. Pluronic F108 was slowly added and reaction mixture was further stirred at 50° C. for 15 minutes. The mixture was cooled to room temperature followed by addition of Colonial SLS, glycerin, sodium saccharin, zinc chloride, methyl salicylate, menthol, 3,3-bis-(4-hydroxy-3-sec-butylphenyl)-1-(3H)-isobenzofuranone, sodium hydroxide, liquid germall plus and remaining deionized water. The reaction mixture was further stirred for 2 hours at room temperature.
Color of the solution: Pink
Color change: Pink to colorless

### Example 15

| Chemical Component | Weight in grams |
|---|---|
| Pluronic F108 | 0.5 |
| Colonial SLS | 1 |
| Glycerin | 8 |
| Sodium saccharin | 0.05 |
| Zinc chloride | 0.005 |
| Methyl salicylate | 0.1 |
| Menthol | 0.1 |
| 3,3-bis-(4-hydroxy-3-nitrophenyl)- | 0.5 |
| 1-(3H)-isobenzofuranone | |
| Sodium hydroxide | 0.08 |
| Deionized water | 89.565 |
| Liquid Germall Plus | 0.1 |

In a beaker, deionized water (50 g) was stirred and heated at 50° C. Pluronic F108 was slowly added and reaction mixture was further stirred at 50° C. for 15 minutes. The mixture was cooled to room temperature followed by addition of Colonial SLS, glycerin, sodium saccharin, zinc chloride, methyl salicylate, menthol, 3,3-bis-(4-hydroxy-3-nitrophenyl)-1-(3H)-isobenzofuranone, sodium hydroxide, liquid germall plus and remaining deionized water. The reaction mixture was further stirred for 2 hours at room temperature.
Color of the solution: Yellow
Color change: Yellow to colorless

### Example 16

| Chemical Component | Weight in grams |
|---|---|
| Pluronic F108 | 0.5 |
| Colonial SLS | 1 |
| Glycerin | 8 |
| Sodium saccharin | 0.05 |
| Zinc chloride | 0.005 |
| Methyl salicylate | 0.1 |
| Menthol | 0.1 |
| m-Nitrophenol | 0.5 |
| Sodium hydroxide | 0.08 |
| Deionized water | 89.565 |
| Liquid Germall Plus | 0.1 |

In a beaker, deionized water (50 g) was stirred and heated at 50° C. Pluronic F108 was slowly added and reaction mixture was further stirred at 50° C. for 15 minutes. The mixture was cooled to room temperature followed by addition of Colonial SLS, glycerin, sodium saccharin, zinc chloride, methyl salicylate, menthol, m-nitrophenol, sodium hydroxide, liquid germall plus and remaining deionized water. The reaction mixture was further stirred for 2 hours at room temperature.
Color of the solution: Golden yellow
Color change: Golden yellow to colorless

### Example 17

| Chemical Component | Weight in grams |
|---|---|
| Scope Mouthwash^{a} | 50 |
| Dye^{b} | 0.5 |
| Sodium hydroxide | 2 equivalent |

Scope Mouthwash^{a}: Ingredients include water, alcohol, glycerin, flavor, polysorbate 80, sodium saccharin, sodium benzoate, cetyl pyridinium chloride, benzoid acid, Blue 1.
Dye^{b}: All the above dyes were used.

In a beaker containing Scope mouthwash was added dye and sodium hydroxide. The reaction mixture was further stirred for 2 hours at room temperature.

Test procedure model: A solution of citric acid was prepared by adding 0.050 g of citric acid to 5 mL of water. The solution was stirred until the citric acid was dissolved. A few drops of the solution were added to the palm of a hand, rubbed over the hand and placed over a 25 mL container with the 10 mL of the above-prepared solution. The mouthwash solution was shaken so that the mouthwash solution was contacted with the wetted palm.

The color remained for approximately 30 seconds during shaking and completely faded in about 1 minute.

Similarly, LISTERINE, CEPACOL or CREST mouthwash compositions can be prepared with indicator dyes of the invention.

The mouthwash composition may be buffered with an appropriate buffer e.g. sodium citrate or phosphate in the pH-range 6-8.5.

The mouth wash can be in non-diluted form (i.e. must be diluted before use).

### TOOTHPASTE EXAMPLES

A toothpaste of the invention (in weight % of the final toothpaste composition) can include the following ingredients:

| | |
|---|---|
| 0-20% | Humectant |
| 0-8% | Surfactant (Gelling agent or binder) |
| 0-40% | Polishing agent |
| 0-70% | Thickners/viscosifiers |
| 0-5% | optional ingredients, i.e., sodium fluoride, enzymes, |
| | disinfecting cleaners, antimicrobial agents, sweetners, |
| | flavors, gums, preservatives etc. |
| 0-20% | Ethanol (or other pharmaceutically acceptable alcohol) |
| 0.01-5% | acid-base indicator of the invention |
| 0-40% | Water |

### Example 1

| Chemical Component | Weight in grams |
|---|---|
| Pluronic F127 | 0.5 |
| Aerosil 200 | 0.1 |
| Methocel A4M (2% solution) | 62.12 |
| Colonial SLS | 1 |
| Glycerin | 10 |
| Sodium saccharin | 0.07 |
| Menthol | 0.2 |
| Sodium fluoride | 0.4 |
| Thymolphthalein | 0.5 |
| Sodium hydroxide | 0.09 |
| Deionized water | 25 |
| Liquid Germall Plus | 0.02 |

In a beaker, deionized water was stirred and heated at 40° C. Pluronic F127 and Aerosil 200 were slowly added and reaction mixture was further stirred at 40° C. for 20 minutes. The mixture was cooled to room temperature followed by addition of Colonial SLS, glycerin, sodium saccharin, menthol, sodium fluoride, thymolphthalein, sodium hydroxide, Methocel A4M and liquid germall plus. The reaction mixture was further stirred for 2 hours at room temperature.
Color of the solution: Blue
Color change: Blue to colorless

### Example 2

| Chemical Component | Weight in grams |
|---|---|
| Pluronic F127 | 0.5 |
| Aerosil 200 | 0.1 |
| Methocel A4M (2% solution) | 62.12 |
| Colonial SLS | 1 |
| Glycerin | 10 |
| Sodium saccharin | 0.07 |
| Menthol | 0.2 |
| Sodium fluoride | 0.4 |
| o-Cresolphthalein | 0.5 |
| Sodium hydroxide | 0.11 |
| Deionized water | 24.98 |
| Liquid Germall Plus | 0.02 |

In a beaker, deionized water was stirred and heated at 40° C. Pluronic F127 and Aerosil 200 were slowly added and reaction mixture was further stirred at 40° C. for 20 minutes. The mixture was cooled to room temperature followed by addition of Colonial SLS, glycerin, sodium saccharin, menthol, sodium fluoride, o-cresolphthalein, sodium hydroxide, Methocel A4M and liquid germall plus. The reaction mixture was further stirred for 2 hours at room temperature.
Color of the solution: Magenta
Color change: Magenta to colorless

### Example 3

| Chemical Component | Weight in grams |
|---|---|
| Pluronic F127 | 0.5 |
| Aerosil 200 | 0.1 |
| Methocel A4M (2% solution) | 62.12 |
| Colonial SLS | 1 |
| Glycerin | 10 |
| Sodium saccharin | 0.07 |
| Menthol | 0.2 |
| Sodium fluoride | 0.4 |
| 3,3-bis-(4-hydroxy-3-phenylphenyl)- | 0.5 |
| 1-(3H)-isobenzofuranone | |
| Sodium hydroxide | 0.08 |
| Deionized water | 25.01 |
| Liquid Germall Plus | 0.02 |

In a beaker, deionized water was stirred and heated at 40° C. Pluronic F127 and Aerosil 200 were slowly added and reaction mixture was further stirred at 40° C. for 20 minutes. The mixture was cooled to room temperature followed by addition of Colonial SLS, glycerin, sodium saccharin, menthol, sodium fluoride, 3,3-bis-(4-hydroxy-3-phenylphenyl)-1-(3H)-isobenzofuranone, sodium hydroxide, Methocel A4M and liquid germall plus. The reaction mixture was further stirred for 2 hours at room temperature.
Color of the solution: Purple
Color change: Purple to colorless

### Example 4

| Chemical Component | Weight in grams |
|---|---|
| Pluronic F127 | 0.5 |
| Aerosil 200 | 0.1 |
| Methocel A4M (2% solution) | 62.12 |
| Colonial SLS | 1 |
| Glycerin | 10 |
| Sodium saccharin | 0.07 |
| Menthol | 0.2 |
| Sodium fluoride | 0.4 |
| 3,3-bis-(4-hydroxy-3,5-diisopropylphenyl)- | 0.5 |
| 1-(3H)-isobenzofuranone | |
| Sodium hydroxide | 0.08 |
| Deionized water | 25.01 |
| Liquid Germall Plus | 0.02 |

In a beaker, deionized water was stirred and heated at 40° C. Pluronic F127 and Aerosil 200 were slowly added and reaction mixture was further stirred at 40° C. for 20 minutes. The mixture was cooled to room temperature followed by addition of Colonial SLS, glycerin, sodium saccharin, menthol, sodium fluoride, 3,3-bis-(4-hydroxy-3,5-diisopropylphenyl)-1-(3H)-isobenzofuranone, sodium hydroxide, Methocel A4M and liquid germall plus. The reaction mixture was further stirred for 2 hours at room temperature.
Color of the solution: Violet
Color change: Violet to colorless

### Example 5

| Chemical Component | Weight in grams |
|---|---|
| Pluronic F127 | 0.5 |
| Aerosil 200 | 0.1 |
| Methocel A4M (2% solution) | 62.12 |
| Colonial SLS | 1 |
| Glycerin | 10 |
| Sodium saccharin | 0.07 |
| Menthol | 0.2 |
| Sodium fluoride | 0.4 |
| 3,3-bis-(4-hydroxy-3,5-dimethoxyphenyl)- | 0.5 |
| 1-(3H)-isobenzofuranone | |
| Sodium hydroxide | 0.09 |
| Deionized water | 25 |
| Liquid Germall Plus | 0.02 |

In a beaker, deionized water was stirred and heated at 40° C. Pluronic F127 and Aerosil 200 were slowly added and reaction mixture was further stirred at 40° C. for 20 minutes. The mixture was cooled to room temperature followed by addition of Colonial SLS, glycerin, sodium saccharin, menthol, sodium fluoride, 3,3-bis-(4-hydroxy-3,5-dimethoxyphenyl)-1-(3H)-isobenzofuranone, sodium hydroxide, Methocel A4M and liquid germall plus. The reaction mixture was further stirred for 2 hours at room temperature.
Color of the solution: Teal
Color change: Teal to colorless

### Example 6

| Chemical Component | Weight in grams |
|---|---|
| Pluronic F127 | 0.5 |
| Aerosil 200 | 0.1 |
| Methocel A4M (2% solution) | 62.12 |
| Colonial SLS | 1 |
| Glycerin | 10 |
| Sodium saccharin | 0.07 |
| Menthol | 0.2 |
| Sodium fluoride | 0.4 |
| 3,3-bis-(4-hydroxy-3,5-dimethylphenyl)- | 0.5 |
| 1-(3H)-isobenzofuranone | |
| Sodium hydroxide | 0.1 |
| Deionized water | 24.99 |
| Liquid Germall Plus | 0.02 |

In a beaker, deionized water was stirred and heated at 40° C. Pluronic F127 and Aerosil 200 were slowly added and reaction mixture was further stirred at 40° C. for 20 minutes. The mixture was cooled to room temperature followed by addition of Colonial SLS, glycerin, sodium saccharin, menthol, sodium fluoride, 3,3-bis-(4-hydroxy-3,5-dimethylphenyl)-1-(3H)-isobenzofuranone, sodium hydroxide, Methocel A4M and liquid germall plus. The reaction mixture was further stirred for 2 hours at room temperature.
Color of the solution: Purple
Color change: Purple to colorless

### Example 7

| Chemical Component | Weight in grams |
|---|---|
| Pluronic F127 | 0.5 |
| Aerosil 200 | 0.1 |
| Methocel A4M (2% solution) | 62.12 |
| Colonial SLS | 1 |
| Glycerin | 10 |
| Sodium saccharin | 0.07 |
| Menthol | 0.2 |
| Sodium fluoride | 0.4 |
| 3,3-bis-(4-hydroxy-3,6-diimethylphenyl)- | 0.5 |
| 1-(3H)-isobenzofuranone | |
| Sodium hydroxide | 0.1 |
| Deionized water | 24.99 |
| Liquid Germall Plus | 0.02 |

In a beaker, deionized water was stirred and heated at 40° C. Pluronic F127 and Aerosil 200 were slowly added and reaction mixture was further stirred at 40° C. for 20 minutes. The mixture was cooled to room temperature followed by addition of Colonial SLS, glycerin, sodium saccharin, menthol, sodium fluoride, 3,3-bis-(4-hydroxy-3,6-diimethylphenyl)-1-(3H)-isobenzofuranone, sodium hydroxide, Methocel A4M and liquid germall plus. The reaction mixture was further stirred for 2 hours at room temperature.
Color of the solution: Blue
Color change: Blue to colorless

### Example 8

| Chemical Component | Weight in grams |
|---|---|
| Pluronic F127 | 0.5 |
| Aerosil 200 | 0.1 |
| Methocel A4M (2% solution) | 62.12 |
| Colonial SLS | 1 |
| Glycerin | 10 |
| Sodium saccharin | 0.07 |
| Menthol | 0.2 |
| Sodium fluoride | 0.4 |
| 3,3-bis-(4-hydroxy-3-ethylphenyl)- | 0.5 |
| 1-(3H)-isobenzofuranone | |
| Sodium hydroxide | 0.1 |
| Deionized water | 24.99 |
| Liquid Germall Plus | 0.02 |

In a beaker, deionized water was stirred and heated at 40° C. Pluronic F127 and Aerosil 200 were slowly added and reaction mixture was further stirred at 40° C. for 20 minutes. The mixture was cooled to room temperature followed by addition of Colonial SLS, glycerin, sodium saccharin, menthol, sodium fluoride, 3,3-bis-(4-hydroxy-3-ethylphenyl)-1-(3H)-isobenzofuranone, sodium hydroxide, Methocel A4M and liquid germall plus. The reaction mixture was further stirred for 2 hours at room temperature.
Color of the solution: Magenta
Color change: Magenta to colorless

### Example 9

| Chemical Component | Weight in grams |
|---|---|
| Pluronic F127 | 0.5 |
| Aerosil 200 | 0.1 |
| Methocel A4M (2% solution) | 62.12 |
| Colonial SLS | 1 |
| Glycerin | 10 |
| Sodium saccharin | 0.07 |
| Menthol | 0.2 |
| Sodium fluoride | 0.4 |
| 3,3-bis-(4-hydroxy-3-isopropylphenyl)- | 0.5 |
| 1-(3H)-isobenzofuranone | |
| Sodium hydroxide | 0.09 |
| Deionized water | 25 |
| Liquid Germall Plus | 0.02 |

In a beaker, deionized water was stirred and heated at 40° C. Pluronic F127 and Aerosil 200 were slowly added and reaction mixture was further stirred at 40° C. for 20 minutes. The mixture was cooled to room temperature followed by addition of Colonial SLS, glycerin, sodium saccharin, menthol, sodium fluoride, 3,3-bis-(4-hydroxy-3-isopropylphenyl)-1-(3H)-isobenzofuranone, sodium hydroxide, Methocel A4M and liquid germall plus. The reaction mixture was further stirred for 2 hours at room temperature.
Color of the solution: Pink
Color change: Pink to colorless

### Example 10

| Chemical Component | Weight in grams |
|---|---|
| Pluronic F127 | 0.5 |
| Aerosil 200 | 0.1 |
| Methocel A4M (2% solution) | 62.12 |
| Colonial SLS | 1 |
| Glycerin | 10 |
| Sodium saccharin | 0.07 |
| Menthol | 0.2 |
| Sodium fluoride | 0.4 |
| 3,3-bis-(4-hydroxy-3-methoxyphenyl)- | 0.5 |
| 1-(3H)-isobenzofuranone | |
| Sodium hydroxide | 0.1 |
| Deionized water | 24.99 |
| Liquid Germall Plus | 0.02 |

In a beaker, deionized water was stirred and heated at 40° C. Pluronic F127 and Aerosil 200 were slowly added and reaction mixture was further stirred at 40° C. for 20 minutes. The mixture was cooled to room temperature followed by addition of Colonial SLS, glycerin, sodium saccharin, menthol, sodium fluoride, 3,3-bis-(4-hydroxy-3-methoxyphenyl)-1-(3H)-isobenzofuranone, sodium hydroxide, Methocel A4M and liquid germall plus. The reaction mixture was further stirred for 2 hours at room temperature.
Color of the solution: Blue
Color change: Blue to colorless

### Example 11

| Chemical Component | Weight in grams |
|---|---|
| Pluronic F127 | 0.5 |
| Aerosil 200 | 0.1 |
| Methocel A4M (2% solution) | 62.12 |
| Colonial SLS | 1 |
| Glycerin | 10 |
| Sodium saccharin | 0.07 |
| Menthol | 0.2 |
| Sodium fluoride | 0.4 |
| 3,3-bis-(4-hydroxy-2,3,5-trimethylphenyl)- | 0.5 |
| 1-(3H)-isobenzofuranone | |
| Sodium hydroxide | 0.09 |
| Deionized water | 25 |
| Liquid Germall Plus | 0.02 |

In a beaker, deionized water was stirred and heated at 40° C. Pluronic F127 and Aerosil 200 were slowly added and reaction mixture was further stirred at 40° C. for 20 minutes. The mixture was cooled to room temperature followed by addition of Colonial SLS, glycerin, sodium saccharin, menthol, sodium fluoride, 3,3-bis-(4-hydroxy-2,3,5-trimethylphenyl)-1-(3H)-isobenzofuranone, sodium hydroxide, Methocel A4M and liquid germall plus. The reaction mixture was further stirred for 2 hours at room temperature.
Color of the solution: Teal
Color change: Teal to colorless

### Example 12

| Chemical Component | Weight in grams |
|---|---|
| Pluronic F127 | 0.5 |
| Aerosil 200 | 0.1 |
| Methocel A4M (2% solution) | 62.12 |
| Colonial SLS | 1 |
| Glycerin | 10 |
| Sodium saccharin | 0.07 |
| Menthol | 0.2 |
| Sodium fluoride | 0.4 |
| Bromo-thymolphthalein | 0.5 |
| Sodium hydroxide | 0.08 |
| Deionized water | 25.01 |
| Liquid Germall Plus | 0.02 |

In a beaker, deionized water was stirred and heated at 40° C. Pluronic F127 and Aerosil 200 were slowly added and reaction mixture was further stirred at 40° C. for 20 minutes. The mixture was cooled to room temperature followed by addition of Colonial SLS, glycerin, sodium saccharin, menthol, sodium fluoride, bromo-thymolphthalein, sodium hydroxide, Methocel A4M and liquid germall plus. The reaction mixture was further stirred for 2 hours at room temperature.
Color of the solution: Teal
Color change: Teal to colorless

### Example 13

| Chemical Component | Weight in grams |
|---|---|
| Pluronic F127 | 0.5 |
| Aerosil 200 | 0.1 |
| Methocel A4M (2% solution) | 62.12 |
| Colonial SLS | 1 |
| Glycerin | 10 |
| Sodium saccharin | 0.07 |
| Menthol | 0.2 |
| Sodium fluoride | 0.4 |
| Bromo-o-cresolphthalein | 0.5 |
| Sodium hydroxide | 0.1 |
| Deionized water | 24.99 |
| Liquid Germall Plus | 0.02 |

In a beaker, deionized water was stirred and heated at 40° C. Pluronic F127 and Aerosil 200 were slowly added and reaction mixture was further stirred at 40° C. for 20 minutes. The mixture was cooled to room temperature followed by addition of Colonial SLS, glycerin, sodium saccharin, menthol, sodium fluoride, bromo-o-cresolphthalein, sodium hydroxide, Methocel A4M and liquid germall plus. The reaction mixture was further stirred for 2 hours at room temperature.
Color of the solution: Purple
Color change: Purple to colorless

### Example 14

| Chemical Component | Weight in grams |
|---|---|
| Pluronic F127 | 0.5 |
| Aerosil 200 | 0.1 |
| Methocel A4M (2% solution) | 62.12 |
| Colonial SLS | 1 |
| Glycerin | 10 |
| Sodium saccharin | 0.07 |
| Menthol | 0.2 |
| Sodium fluoride | 0.4 |
| 3,3-bis-(4-hydroxy-3-sec-butylphenyl)- | 0.5 |
| 1-(3H)-isobenzofuranone | |
| Sodium hydroxide | 0.08 |
| Deionized water | 25.01 |
| Liquid Germall Plus | 0.02 |

In a beaker, deionized water was stirred and heated at 40° C. Pluronic F127 and Aerosil 200 were slowly added and reaction mixture was further stirred at 40° C. for 20 minutes. The mixture was cooled to room temperature followed by addition of Colonial SLS, glycerin, sodium saccharin, menthol, sodium fluoride, 3,3-bis-(4-hydroxy-3-sec-butylphenyl)-1-(3H)-isobenzofuranone, sodium hydroxide, Methocel A4M and liquid germall plus. The reaction mixture was further stirred for 2 hours at room temperature.
Color of the solution: Pink
Color change: Pink to colorless

### Example 15

| Chemical Component | Weight in grams |
|---|---|
| Pluronic F127 | 0.5 |
| Aerosil 200 | 0.1 |
| Methocel A4M (2% solution) | 62.12 |
| Colonial SLS | 1 |
| Glycerin | 10 |
| Sodium saccharin | 0.07 |
| Menthol | 0.2 |
| Sodium fluoride | 0.4 |
| 3,3-bis-(4-hydroxy-3-nitrophenyl)- | 0.5 |
| 1-(3H)-isobenzofuranone | |
| Sodium hydroxide | 0.08 |
| Deionized water | 25.01 |
| Liquid Germall Plus | 0.02 |

In a beaker, deionized water was stirred and heated at 40° C. Pluronic F127 and Aerosil 200 were slowly added and reaction mixture was further stirred at 40° C. for 20 minutes. The mixture was cooled to room temperature followed by addition of Colonial SLS, glycerin, sodium saccharin, menthol, sodium fluoride, 3,3-bis-(4-hydroxy-3-nitrophenyl)-1-(3H)-isobenzofuranone, sodium hydroxide, Methocel A4M and liquid germall plus. The reaction mixture was further stirred for 2 hours at room temperature.
Color of the solution: Yellow
Color change: Yellow to colorless

### Example 16

| Chemical Component | Weight in grams |
|---|---|
| Pluronic F127 | 0.5 |
| Aerosil 200 | 0.1 |
| Methocel A4M (2% solution) | 62.12 |
| Colonial SLS | 1 |
| Glycerin | 10 |
| Sodium saccharin | 0.07 |
| Menthol | 0.2 |
| Sodium fluoride | 0.4 |
| m-Nitrophenol | 0.5 |
| Sodium hydroxide | 0.08 |
| Deionized water | 25.01 |
| Liquid Germall Plus | 0.02 |

In a beaker, deionized water was stirred and heated at 400C. Pluronic F 127 and Aerosil 200 were slowly added and reaction mixture was further stirred at 40° C. for 20 minutes. The mixture was cooled to room temperature followed by addition of Colonial SLS, glycerin, sodium saccharin, menthol, sodium fluoride, m-nitrophenol, sodium hydroxide, Methocel A4M and liquid germall plus. The reaction mixture was further stirred for 2 hours at room temperature.
Color of the solution: Golden yellow
Color change: Golden yellow to colorless

### Example 17

| Chemical Component | Weight in grams |
|---|---|
| Commercial Toothpaste^{a,b,c} | 25 |
| Dye^{b} | 0.25 |
| Sodium hydroxide | 2 equivalent |

Commercial Toothpaste^{a}include but not limited to Colgate and Crest toothpaste
Commercial Toothpaste^{b}Colgate ingredients include sodium monofluoro phosphate, dicalcium phosphate dehydrate, water, glycerin, sodium lauryl sulfate, cellulose gum, flavor, tetrasodium pyrophosphate, sodium saccharin.
Commercial Toothpaste^{c}Crest ingredients include sodium fluoride, water, hydrated silica, sorbitol, glycerin, tetrapotassium pyrophosphate, PEG-6, disodium pyrophosphate, tetrasodium pyrophosphate, flavor, sodium lauryl sulfate, xanthum gum, sodium saccharin, carbomer 956, polysorbate 80, sodium benzoate, cetyl pyridinium chloride, benzoid acid, domiphen bromide, titanium dioxide, Blue 1, Yellow 5.
Dye^{b}: All the above dyes were used.

In a beaker containing commercial toothpaste was added dye and sodium hydroxide. The reaction mixture was further stirred for 2 hours at room temperature.

Test Procedure:

Colored toothpaste from any of the above examples was taken on a toothbrush. Artificial teeth were then brushed with the toothbrush containing toothpaste from the above examples. Water was added couple of times during the brushing. The respective color disappears in 30 seconds to 1 minute

Synthesis of Acid-base Indicators:

### Example 1

### Synthesis of 3,3-bis-(4-hydroxy-3-isopropylphenyl)-1-(3H)-isobenzofuranone

A mixture of 2-isopropylphenol (0.2M), phthalic anhydride (0.1M), polyphosphoric acid (0.25M), and zinc chloride (0.01M), was stirred and heated at 100° C. for 3 hours. The reaction mixture was cooled to room temperature and added to ice-water mixture when the product precipitated. The product was filtered, thoroughly washed with water and dried. Recrystallization from ethanol with charcoal treatment furnished pure 3,3-bis-(4-hydroxy-3-isopropylphenyl)-1-(3H)-isobenzofuranone in 96% yield.

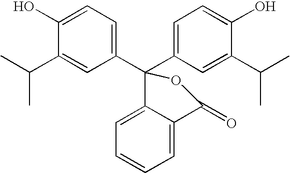

### Example2

### Synthesis of 3,3-bis-(4-hydroxy-3,5-diisopropylphenyl)-1-(3H)-isobenzofuranone

A mixture of 2,6-diisopropylphenol (0.2M), phthalic anhydride (0.1M), polyphosphoric acid (0.25M), and zinc chloride (0.01M), was stirred and heated at 100° C. for 3 hours. The reaction mixture was cooled to room temperature and added to ice-water mixture when the product precipitated. The product was filtered, thoroughly washed with water and dried. Recrystallization from ethanol with charcoal treatment furnished pure 3,3-bis-(4-hydroxy-3,5-diisopropylphenyl)-1-(3H)-isobenzofuranone in 98% yield.

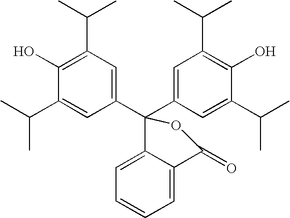

### Example 3

### Synthesis of 3,3-bis-(4-hydroxy-2-nitrophenyl)-1-(3H)-isobenzofuranone

A mixture of 3-nitrophenol (0.2M), phthalic anhydride (0.1M), polyphosphoric acid (0.25M), and zinc chloride (0.01M), was stirred and heated at 100° C. for 3 hours. The reaction mixture was cooled to room temperature and added to ice-water mixture when the product precipitated. The product was filtered, thoroughly washed with water and dried. Recrystallization from ethanol with charcoal treatment furnished pure 3,3-bis-(4-hydroxy-2-nitrophenyl)-1-(3H)-isobenzofuranone in 81% yield.

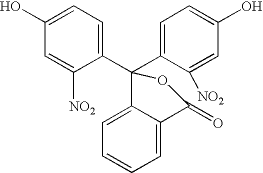

### Example 4

### Synthesis of 3,3-bis-(4-hydroxy-3-nitrophenyl)-1-(3H)-isobenzofuranone

A mixture of 2-nitrophenol (0.2M), phthalic anhydride (0.1M), polyphosphoric acid (0.25M), and zinc chloride (0.01M), was stirred and heated at 100° C. for 3 hours. The reaction mixture was cooled to room temperature and added to ice-water mixture when the product precipitated. The product was filtered, thoroughly washed with water and dried. Recrystallization from ethanol with charcoal treatment furnished pure 3,3-bis-(4-hydroxy-3-nitrophenyl)-1-(3H)-isobenzofuranone in 89% yield.

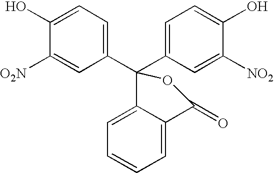

### Example 5

### Synthesis of 3,3-bis-14-hydroxy-2-(N,N-diethylamino)phenyl-1-(3H)-isobenzofuranone

A mixture of 3-(N,N-diethylamino)phenol (0.2M), phthalic anhydride (0.1M), polyphosphoric acid (0.25M), and zinc chloride (0.01M), was stirred and heated at 100° C. for 3 hours. The reaction mixture was cooled to room temperature and added to ice-water mixture when the product precipitated. The product was filtered, thoroughly washed with water and dried. Recrystallization from ethanol with charcoal treatment furnished pure 3,3-bis-[4-hydroxy-2-(N,N-diethylamino)phenyl]-1-(3H)-isobenzofuranone in 93% yield.

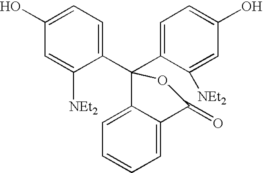

### Example 6

### Synthesis of 3,3-bis-(4-hydroxy-3-ethylphenyl)-1-(3H)-isobenzofuranone

A mixture of 2-ethylphenol (0.2M), phthalic anhydride (0.1M), polyphosphoric acid (0.25M), and zinc chloride (0.01M), was stirred and heated at 100° C. for 3 hours. The reaction mixture was cooled to room temperature and added to ice-water mixture when the product precipitated. The product was filtered, thoroughly washed with water and dried. Recrystallization from ethanol with charcoal treatment furnished pure 3,3-bis-(4-hydroxy-3-ethylphenyl)-1-(3H)-isobenzofuranone in 92% yield.

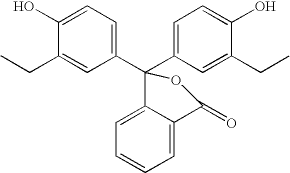

### Example 7

### Synthesis of 3,3-bis-(4-hydroxy-3-ethoxyphenyl)-1-(3H)-isobenzofuranone

A mixture of 2-ethoxyphenol (0.2M), phthalic anhydride (0.1M), polyphosphoric acid (0.25M), and zinc chloride (0.01M), was stirred and heated at 100° C. for 3 hours. The reaction mixture was cooled to room temperature and added to ice-water mixture when the product precipitated. The product was filtered, thoroughly washed with water and dried. Recrystallization from ethanol with charcoal treatment furnished pure 3,3-bis-(4-hydroxy-3-ethoxyphenyl)-1-(3H)-isobenzofuranone in 85% yield.

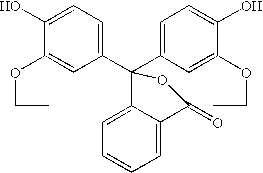

### Example 8

### Synthesis of 3,3-bis-(4-hydroxy-3-acetamidophenyl)-1-(3H)-isobenzofuranone

A mixture of 2-acetamidophenol (0.2M), phthalic anhydride (0.1M), polyphosphoric acid (0.25M), and zinc chloride (0.01M), was stirred and heated at 100° C. for 3 hours. The reaction mixture was cooled to room temperature and added to ice-water mixture when the product precipitated. The product was filtered, thoroughly washed with water and dried. Recrystallization from ethanol with charcoal treatment furnished pure 3,3-bis-(4-hydroxy-3-acetamidophenyl)-1-(3H)-isobenzofuranone in 83% yield.

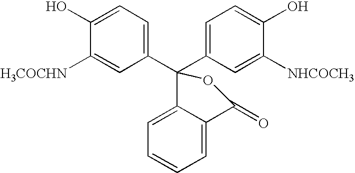

### Example 9

### Synthesis of 3,3-bis-(4-hydroxy-6-methyl-3-nitrophenyl)-1-(3H)-isobenzofuranone

A mixture of 5-methyl-2-nitrophenol (0.2M), phthalic anhydride (0.1M), polyphosphoric acid (0.25M), and zinc chloride (0.01M), was stirred and heated at 100° C. for 3 hours. The reaction mixture was cooled to room temperature and added to ice-water mixture when the product precipitated. The product was filtered, thoroughly washed with water and dried. Recrystallization from ethanol with charcoal treatment furnished pure 3,3-bis-(4-hydroxy-6-methyl-3-nitrophenyl)-1-(3H)-isobenzofuranone in 81% yield.

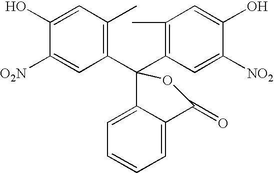

### Example 10

### Synthesis of 3,3-bis-(4-hydroxy-6-methyl-5-quinotin-1-yl)-1-(3H)-isobenzofuranone

A mixture of 8-hydroxyquinaldine (0.2M), phthalic anhydride (0.1M), polyphosphoric acid (0.25M), and zinc chloride (0.01M), was stirred and heated at 100° C. for 3 hours. The reaction mixture was cooled to room temperature and added to ice-water mixture when the product precipitated. The product was filtered, thoroughly washed with water and dried. Recrystallization from ethanol with charcoal treatment furnished pure 3,3-bis-(4-hydroxy-6-methyl-5-quinolin-1-yl)-1-(3H)-isobenzofuranone in 88% yield.

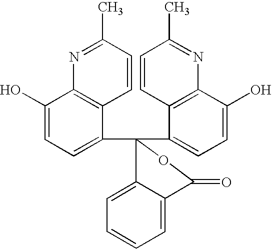

### Example 11

### Synthesis of 3,3-bis-(4-hydroxy-3-pyridin-1-yl)-1-(3H)-isobenzofuranone

A mixture of 2-hydroxypyridine (0.2M), phthalic anhydride (0.1M), polyphosphoric acid (0.25M), and zinc chloride (0.01M), was stirred and heated at 100° C. for 3 hours. The reaction mixture was cooled to room temperature and added to ice-water mixture when the product precipitated. The product was filtered, thoroughly washed with water and dried. Recrystallization from ethanol with charcoal treatment furnished pure 3,3-bis-(4-hydroxy-3-pyridin-1-yl)-1-(3H)-isobenzofuranone in 80% yield.

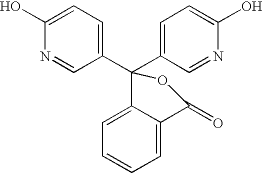

### Example 12

### Synthesis of 3,3-bis-(4-hydroxy-2-pyridin-1-yl)-1-(3H)-isobenzofuranone

A mixture of 3-hydroxypyridine (0.2M), phthalic anhydride (0.1M), polyphosphoric acid (0.25M), and zinc chloride (0.01M), was stirred and heated at 100° C. for 3 hours. The reaction mixture was cooled to room temperature and added to ice-water mixture when the product precipitated. The product was filtered, thoroughly washed with water and dried. Recrystallization from ethanol with charcoal treatment furnished pure 3,3-bis-(4-hydroxy-2-pyridin-1-yl)-1-(3H)-isobenzofuranone in 82% yield.

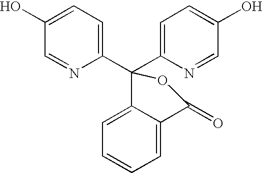

### Example 13

### Synthesis of 3,3-bis-(4-hydroxy-3-phenylphenyl)-1-(3H)-isobenzofuranone

A mixture of 2-phenylphenol (0.133 mol), phthalic anhydride (0.074 mol), reaction medium (0.416 mol), and Lewis acid (0.029 mol), was stirred and heated at 90° C. for 5 hours. The reaction mixture was cooled to room temperature and added to ice-water mixture when the product precipitated. The product was filtered, thoroughly washed with water and dried. Recrystallization from methanol with charcoal treatment furnished pure 3,3-bis-(4-hydroxy-3-phenylphenyl)-1-(3H)-isobenzofuranone as white crystals in 94% yield. ¹H-NMR (DMSO-d₆, 300 MHz): δ 9.89 (s, 2H, 2OH), 6.97-7.18 (m, 6H, aromatic), 7.26-7.47 (m, 10H, aromatic), 7.63-7.92 (m, 4H, aromatic) ppm. Mass spectra: m/z 470 (M⁺).

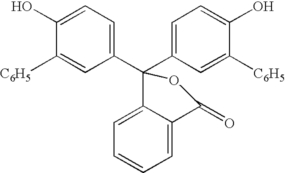

### Example 14

### Synthesis of 3,3-bis-(4-hydroxy-3,5-diisopropylphenyl)-1-(3H)-isobenzofuranone

A mixture of 2,6-diisopropylphenol (0.133 mol), phthalic anhydride (0.074 mol), reaction medium (0.416 mol), and Lewis acid (0.029 mol), was stirred and heated at 90° C. for 5 hours. The reaction mixture was cooled to room temperature and added to ice-water mixture when the product precipitated. The product was filtered, thoroughly washed with water and dried. Recrystallization from methanol with charcoal treatment furnished pure 3,3-bis-(4-hydroxy-3,5-diisopropylphenyl)-1-(3H)-isobenzofuranone as white crystals in 89% yield. IR (KBr): 3506, 1734, 1609 cm⁻¹. ¹H-NMR (DMSO-d₆): δ 9.56 (s, 2H, 2OH), 1.02-1.05 (dd, 24H, 8CH₃), 3.22-3.31 (heptate, 4H, 4CH), 6.74-7.00 (m, 4H, aromatic), 7.59-7.92 (m, 4H, aromatic) ppm. Mass spectra: m/z 486 (M⁺).

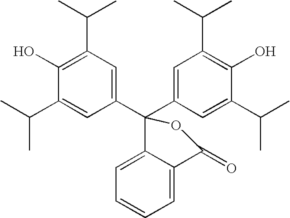

### Example 15

### Synthesis of 3,3-bis-(4-hydroxy-3,5-dimethoxyphenyl)-1-(3H)-isobenzofuranone

A mixture of 2,6-dimethoxyphenol (0.133 mol), phthalic anhydride (0.074 mol), reaction medium (0.416 mol), and Lewis acid (0.029 mol), was stirred and heated at 90° C. for 5 hours. The reaction mixture was cooled to room temperature and added to ice-water mixture when the product precipitated. The product was filtered, thoroughly washed with water and dried. Recrystallization from methanol with charcoal treatment furnished pure 3,3-bis-(4-hydroxy-3,5-dimethoxyphenyl)-1-(3H)-isobenzofuranone as white crystals in 84% yield. IR (KBr): 3388, 1769, 1606, 1369 cm⁻¹. ¹H-NMR (DMSO-d₆): δ 8.71 (s, 2H, 2OH), 3.66 (s, 12H, 4OCH₃), 7.65-7.68 (m, 4H, aromatic), 7.83-7.96 (m, 4H, aromatic) ppm. Mass spectra: m/z 438 (M⁺).

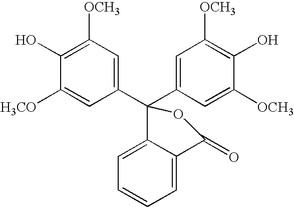

### Example 16

### Synthesis of 3,3-bis-(4-hydroxy-3,5-dimethylphenyl)-1-(3H)-isobenzofuranone

A mixture of 2,6-dimethylphenol (0.133 mol), phthalic anhydride (0.074 mol), reaction medium (0.416 mol), and Lewis acid (0.029 mol), was stirred and heated at 90° C. for 5 hours. The reaction mixture was cooled to room temperature and added to ice-water mixture when the product precipitated. The product was filtered, thoroughly washed with water and dried. Recrystallization from methanol with charcoal treatment furnished pure 3,3-bis-(4-hydroxy-3,5-dimethylphenyl)-1-(3H)-isobenzofuranone as white crystals in 91% yield. IR (KBr): 3582, 3386, 1746, 1605 cm⁻¹. ¹H-NMR (DMSO-d₆): δ 8.45 (s, 2H, 2OH), 2.10 (s, 12H, 4CH₃), 7.58-7.63 (m, 4H, aromatic), 7.78-7.87 (m, 4H, aromatic) ppm. Mass spectra: m/z 374 (M⁺).

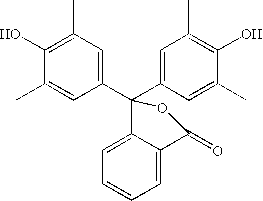

### Example 17

### Synthesis of 3,3-bis-(4-hydroxy-3,6-diimethylphenyl)-1-(3H)-isobenzofuranone

A mixture of 2,5-dimethylphenol (0.133 mol), phthalic anhydride (0.074 mol), reaction medium (0.416 mol), and Lewis acid (0.029 mol), was stirred and heated at 90° C. for 5 hours. The reaction mixture was cooled to room temperature and added to ice-water mixture when the product precipitated. The product was filtered, thoroughly washed with water and dried. Recrystallization from methanol with charcoal treatment furnished pure 3,3-bis-(4-hydroxy-3,6-diimethylphenyl)-1-(3H)-isobenzofuranone as pale yellow crystals in 85% yield. IR (KBr): 3393, 1729, 1611 cm⁻¹. ¹H-NMR (DMSO-d₆): δ 9.40 (s, 2H, 2OH), 1.95 (s, 12H, 4CH₃), 6.59-6.63 (m, 4H, aromatic), 7.46-7.91 (m, 4H, aromatic) ppm. Mass spectra: m/z 374 (M⁺).

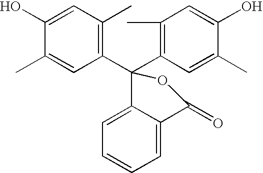

### Example 18

### Synthesis of 3,3-bis-(4-hydroxy-3-ethylphenyl)-1-(3n)-isobenzofuranone

A mixture of 2-ethylphenol (0.133 mol), phthalic anhydride (0.074 mol), reaction medium (0.416 mol), and Lewis acid (0.029 mol), was stirred and heated at 90° C. for 5 hours. The reaction mixture was cooled to room temperature and added to ice-water mixture when the product precipitated. The product was filtered, thoroughly washed with water and dried. Recrystallization from ethyl acetate:petroleum ether (1:1) with charcoal treatment furnished pure 3,3-bis-(4-hydroxy-3-ethylphenyl)-1-(3H)-isobenzofuranone as white crystals in 81% yield. IR (KBr): 3389, 1783, 1718, 1605 cm⁻¹. ¹H-NMR (DMSO-d₆): δ 9.54 (s, 2H, 2OH), 2.43-2.50 (q, 4H, 2CH₂), 1.00-1.05 (t, 6H, 2CH₃), 6.74-6.96 (m, 6H, aromatic), 7.57-7.89 (m, 4H, aromatic) ppm. Mass spectra: m/z 374 (M⁺).

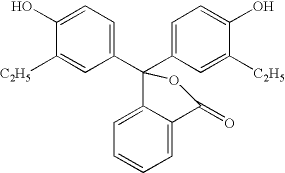

### Example 19

### Synthesis of 3,3-bis-(4-hydroxy-3-isopropylphenyl)-1-(3H)-isobenzofuranone

A mixture of 2-isopropylphenol (0.133 mol), phthalic anhydride (0.074 mol), reaction medium (0.416 mol), and Lewis acid (0.029 mol), was stirred and heated at 90° C. for 5 hours. The reaction mixture was cooled to room temperature and added to ice-water mixture when the product precipitated. The product was filtered, thoroughly washed with water and dried. Recrystallization from methanol:water (1:1) with charcoal treatment furnished pure 33,3-bis-(4-hydroxy-3-isopropylphenyl)-1-(3H)-isobenzofuranone as white crystals in 83% yield. IR (KBr): 3383, 1733, 1608 cm⁻¹. ¹H-NMR (DMSO-d₆): δ 9.57 (s, 2H, 2OH), 1.05-1.07 (dd, 12H, 4CH₃), 3.11-3.18 (heptate, 2H, 2CH), 6.75-7.01 (m, 6H, aromatic), 7.59-7.90 (m, 4H, aromatic) ppm. Mass spectra: m/z 402 (M⁺).

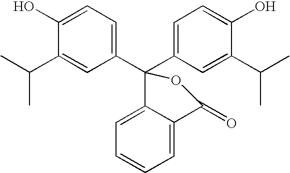

### Example 20

### Synthesis of 3,3-bis-(4-hydroxy-3-methoxyphenyl)-1-(3H)-isobenzofuranone

A mixture of 2-methoxyphenol (0.133 mol), phthalic anhydride (0.074 mol), reaction medium (0.416 mol), and Lewis acid (0.029 mol), was stirred and heated at 90° C. for 5 hours. The reaction mixture was cooled to room temperature and added to ice-water mixture when the product precipitated. The product was filtered, thoroughly washed with water and dried. Recrystallization from methanol with charcoal treatment furnished pure 3,3-bis-(4-hydroxy-3-methoxyphenyl)-1-(3H)-isobenzofuranone as white crystals in 79% yield. IR (KBr): 3517, 1747, 1701, 1279 cm⁻¹. ¹H-NMR (DMSO-d₆): δ 9.27 (s, 2H, 2OH), 3.66 (s, 6H, 2OCH₃), 6.65-6.78 (m, 6H, aromatic), 7.61-7.90 (m, 4H, aromatic) ppm. Mass spectra: m/z 378 (M⁺).

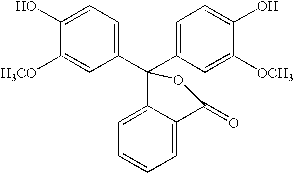

### Example 21

### Synthesis of 3,3-bis-(4-hydroxy-2,3,5-trimethylphenyl)-1-(3H)-isobenzofuranone

A mixture of 2,3,6-trimethylphenol (0.133 mol), phthalic anhydride (0.074 mol), reaction medium (0.416 mol), and Lewis acid (0.029 mol), was stirred and heated at 90° C. for 5 hours. The reaction mixture was cooled to room temperature and added to ice-water mixture when the product precipitated. The product was filtered, thoroughly washed with water and dried. Recrystallization from methanol with charcoal treatment furnished pure 3,3-bis-(4-hydroxy-2,3,5-trimethylphenyl)-1-(3H)-isobenzofuranone as white crystals in 73% yield. IR (KBr): 3510, 3390, 1746, 1609, cm⁻¹. ¹H-NMR (DMSO-d₆): δ 9.44 (s, 2H, 2OH), 2.05 (s, 18H, 6CH₃), 6.55 (s, 2H, aromatic), 7.46-7.90 (m, 4H, aromatic) ppm. Mass spectra: m/z 402 (M⁺).

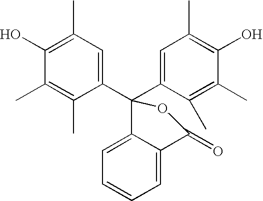

### Example 22

### Synthesis of 3,3-bis-(4-hydroxy-3-sec-butylphenyl)-1-(3H)-isobenzofuranone

A mixture of 2-sec-butylphenol (0.133 mol), phthalic anhydride (0.074 mol), reaction medium (0.416 mol), and Lewis acid (0.029 mol), was stirred and heated at 90° C. for 5 hours. The reaction mixture was cooled to room temperature and added to ice-water mixture when the product precipitated. The product was filtered, thoroughly washed with water and dried. Recrystallization from methanol with charcoal treatment furnished pure 3,3-bis-(4-hydroxy-3-sec-butylphenyl)-1-(3H)-isobenzofuranone as white crystals in 77% yield. IR (KBr): 3400, 1722, 1607 cm⁻¹. ¹H-NMR (DMSO-d₆): δ 9.50 (s, 2H, 2OH), 0.80 (t, 6H, 2CH₃), 1.35-1.39 (p, 4H, 2CH₂), 1.22 (d, 6H, 2CH₃), 2.89-2.97 (sextate, 2H, 2CH), 6.73-6.93 (m, 6H, aromatic), 7.59-7.90 (m, 4H, aromatic) ppm. Mass spectra: m/z 430 (M⁺).

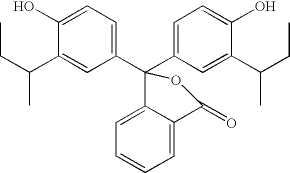

### Example 23

### Synthesis of 3,3-bis-(4-hydroxy-3-nitrophenyl)-1-(3H)-isobenzofuranone

A mixture of phenolphthalein (0.062 mol) in acetic acid (290 mL) was stirred at 15° C. Concentrated nitric acid (0.136 mol, 65%) in acetic acid (10 mL) was slowly added to stirring mixture at 15° C. The reaction mixture was further stirred for 6 hours at room temperature and added to ice-water mixture when the yellow colored product precipitated. The product was filtered, thoroughly washed with water and dried. Recrystallization from ethanol with charcoal treatment furnished pure 3,3-bis-(4-hydroxy-3-nitrophenyl)-1-(3H)-isobenzofuranone as pale yellow crystals in 78% yield. IR (KBr): 3262, 1766, 1627, 1538, 1423 cm⁻¹. ¹H-NMR (DMSO-d₆): δ 9.67 (s, 2H, 2OH), 6.71-7.16 (m, 6H, aromatic), 7.46-7.98 (m, 4H, aromatic) ppm. Mass spectra: m/z 408 (M⁺). 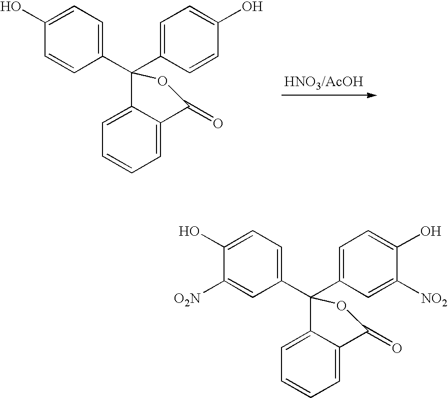
Synthesis of Disodium Salts of Acid-base Indicators for Water Based Systems:

### Example 1

### Synthesis of disodium salt of 3,3-bis-(4-hydroxy-3-isopropylphenyl)-1-(3H1)-isobenzofuranone

A mixture of 3,3-bis-(4-hydroxy-3-isopropylphenyl)-1-(3H)-isobenzofuranone (0.01M) in ethanol (50 mL, 85%) was stirred followed by addition of sodium hydroxide (0.02M) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 98% yield.

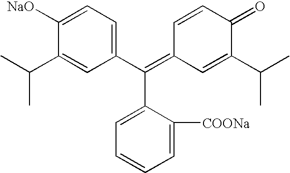

### Example 2

### Synthesis of disodium salt of 3,3-bis-(4-hydroxy-3,5-diisopropylphenyl)-1-(3H)-isobenzofuranone

A mixture of 3,3-bis-(4-hydroxy-3,5-diisopropylphenyl)-1-(3H)-isobenzofuranone (0.01M) in ethanol (50 mL, 85%) was stirred followed by addition of sodium hydroxide (0.02M) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 94% yield.

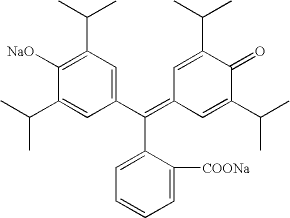

### Example 3

### Synthesis of disodium salt of 3,3-bis-(4-hydroxy-2-nitrophenyl)-1-(3H)-isobenzofuranone

A mixture of 3,3-bis-(4-hydroxy-2-nitrophenyl)-1-(3H)-isobenzofuranone (0.01M) in ethanol (50 mL, 85%) was stirred followed by addition of sodium hydroxide (0.02M) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 88% yield.

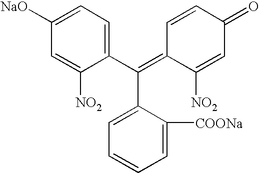

### Example 4

### Synthesis of disodium salt of 3,3-bis-(4-hydroxy-3-nitrophenyl)-1-(3H1)-isobenzofuranone

A mixture of 3,3-bis-(4-hydroxy-3-nitrophenyl)-1-(3H)-isobenzofuranone (0.01M) in ethanol (50 mL, 85%) was stirred followed by addition of sodium hydroxide (0.02M) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 91% yield.

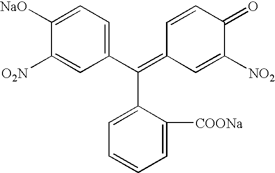

### Example 5

### Synthesis of disodium salt of 3,3-bis-[4-hydroxy-2-(N,N-diethylamino)phenyl]-1-(3H)-isobenzofuranone

A mixture of 3,3-bis-[4-hydroxy-2-(N,N-diethylamino)phenyl]-1-(3H)-isobenzofuranone (0.01 M) in ethanol (50 mL, 85%) was stirred followed by addition of sodium hydroxide (0.02M) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure, disodium salt in 89% yield.

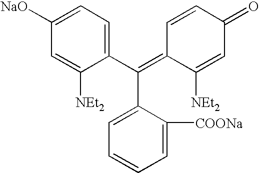

### Example 6

### Synthesis of disodium salt of 3,3-bis-(4-hydroxy-3-ethylphenyl)-1-(3H)-isobenzofuranone

A mixture of 3,3-bis-(4-hydroxy-3-ethylphenyl)-1-(3H)-isobenzofuranone (0.01M) in ethanol (50 mL, 85%) was stirred followed by addition of sodium hydroxide (0.02M) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 97% yield.

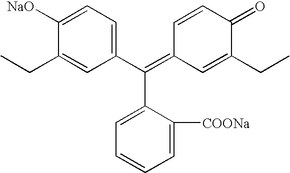

### Example 7

### Synthesis of disodium salt of 3,3-bis-(4-hydroxy-3-ethoxyphenyl)-1-(3H1)-isobenzofuranone

A mixture of 3,3-bis-(4-hydroxy-3-ethoxyphenyl)-1-(3H)-isobenzofuranone (0.01M) in ethanol (50 mL, 85%) was stirred followed by addition of sodium hydroxide (0.02M) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 94% yield.

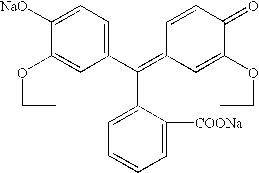

### Example 8

### Synthesis of disodium salt of 3,3-bis-(4-hydroxy-3-acetamidophenyl)-1-(3H)-isobenzofuranone

A mixture of 3,3-bis-(4-hydroxy-3-acetamidophenyl)-1-(3H)-isobenzofuranone (0.01M) in ethanol (50 mL, 85%) was stirred followed by addition of sodium hydroxide (0.02M) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 92% yield.

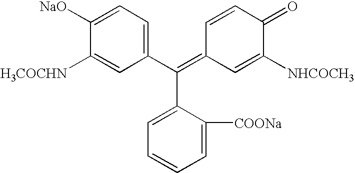

### Example 9

### Synthesis of disodium salt of 3,3-bis-(4-hydroxy-6-methyl-3-nitrophenyl)-1-(3H)-isobenzofuranone

A mixture of 3,3-bis-(4-hydroxy-6-methyl-3-nitrophenyl)-1-(3H)-isobenzofuranone (0.01M) in ethanol (50 mL, 85%) was stirred followed by addition of sodium hydroxide (0.02M) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 97% yield.

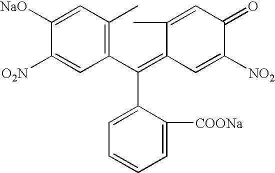

### Example 10

### Synthesis of disodium salt of 3,3-bis-(4-hydroxy-6-methyl-5-quinolin-1-yl)-1-(3H)-isobenzofuranone

A mixture of 3,3-bis-(4-hydroxy-6-methyl-5-quinolin-1-yl)-1-(3H)-isobenzofuranone (0.01M) in ethanol (50 mL, 85%) was stirred followed by addition of sodium hydroxide (0.02M) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 94% yield.

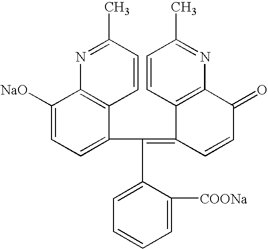

### Example 11

### Synthesis of disodium salt of 3,3-bis-(4-hydroxy-3-pyridin-1-yl)-1-(3H)-isobenzofuranone

A mixture of 3,3-bis-(4-hydroxy-3-pyridin-1-yl)-1-(3H)-isobenzofuranone (0.01M) in ethanol (50 mL, 85%) was stirred followed by addition of sodium hydroxide (0.02M) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 81% yield.

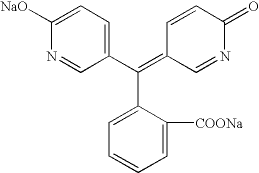

### Example 12

### Synthesis of disodium salt of 3,3-bis-(4-hydroxy-2-pyridin-1-yl)-1-(3H)-isobenzofuranone

A mixture of 3,3-bis-(4-hydroxy-2-pyridin-1-yl)-1-(3H)-isobenzofuranone (0.01M) in ethanol (50 mL, 85%) was stirred followed by addition of sodium hydroxide (0.02M) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 84% yield.

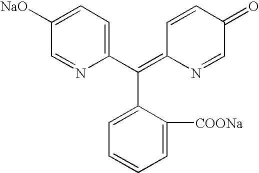

### Example 13

### Synthesis of disodium salt of 3,3-bis-(4-hydroxy-3-phenylphenyl)-1-(3H)-isobenzofuranone

A mixture of 3,3-bis-(4-hydroxy-3-phenylphenyl)-1-(3H)-isobenzofuranone (0.01 mol) in ethanol (50 mL, 85%) was stirred followed by addition of sodium hydroxide (0.02 mol) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 96% yield. ¹H-NMR (DMSO-d₆, 300 MHz): δ 6.25-6.74 (m, 6H, aromatic), 6.88-7.45 (m, 10H, aromatic), 7.53-7.84 (m, 4H, aromatic) ppm. Mass spectra: m/z 514 (M⁺).

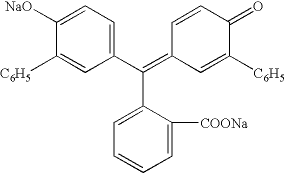

### Example 14

### Synthesis of disodium salt of 3,3-bis-(4-hydroxy-3,5-diisopropylphenyl)-1-(3H)-isobenzofuranone

A mixture of 3,3-bis-(4-hydroxy-3,5-diisopropylphenyl)-1-(3H)-isobenzofuranone (0.01 mol) in ethanol (50 mL, 85%) was stirred followed by addition of sodium hydroxide (0.02 mol) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 92% yield. ¹H-NMR (DMSO-d₆): δ 1.00-1.21 (dd, 24H, 8CH₃), 3.06-3.36 (heptate, 4H, 4CH), 6.74-6.96 (m, 4H, aromatic), 7.05-7.83 (m, 4H, aromatic) ppm. Mass spectra: m/z 530 (M⁺).

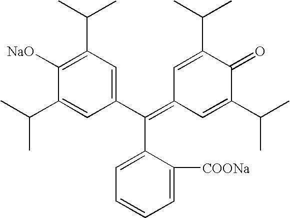

### Example 15

### Synthesis of disodium salt of 3,3-bis-(4-hydroxy-3,5-dimethoxyphenyl)-1-(3H)-isobenzofuranone

A mixture of 3,3-bis-(4-hydroxy-3,5-dimethoxyphenyl)-1-(3H)-isobenzofuranone (0.01 mol) in ethanol (50 mL, 85%) was stirred followed by addition of sodium hydroxide (0.02 mol) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 90% yield. ¹H-NMR (DMSO-d₆): δ 3.61 (s, 12H, 40CH₃), 6.45-6.52 (m, 4H, aromatic), 7.04-7.78 (m, 4H, aromatic) ppm. Mass spectra: m/z 482 (M⁺).

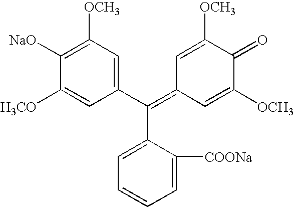

### Example 16

### Synthesis of disodium salt of 3,3-bis-(4-hydroxy-3,5-dimethylphenyl)-1-(3H)-isobenzofuranone

A mixture of 3,3-bis-(4-hydroxy-3,5-dimethylphenyl)-1-(3H)-isobenzofuranone (0.01 mol) in ethanol (50 mL, 85%) was stirred followed by addition of sodium hydroxide (0.02 mol) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 95% yield. ¹H-NMR (DMSO-d₆): δ 2.11 (s, 12H, 4CH₃), 6.81-6.87 (m, 4H, aromatic), 7.23-7.84 (m, 4H, aromatic) ppm. Mass spectra: m/z 418 (M⁺).

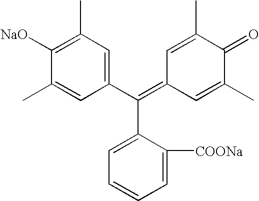

### Example 17

### Synthesis of disodium salt of 3,3-bis-(4-hydroxy-3,6-diimethylphenyl)-1-(3H)-isobenzofuranone

A mixture of 3,3-bis-(4-hydroxy-3,6-diimethylphenyl)-1-(3H)-isobenzofuranone (0.01 mol) in ethanol (50 mL, 85%) was stirred followed by addition of sodium hydroxide (0.02 mol) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 88% yield. ¹H-NMR (DMSO-d₆): δ 2.01 (s, 12H, 4CH₃), 6.04-6.82 (m, 4H, aromatic), 7.10-7.72 (m, 4H, aromatic) ppm. Mass spectra: m/z 418 (M⁺).

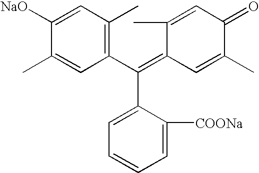

### Example 18

### Synthesis of disodium salt of 3,3-bis-(4-hydroxy-3-ethylphenyl)-1-(3H)-isobenzofuranone

A mixture of 3,3-bis-(4-hydroxy-3-ethylphenyl)-1-(3H)-isobenzofuranone (0.01 mol) in ethanol (50 mL, 85%) was stirred followed by addition of sodium hydroxide (0.02 mol) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 86% yield. ¹H-NMR (DMSO-d₆): δ 2.30-2.51 (q, 4H, 2CH₂), 1.00-1.10 (t, 6H, 2CH₃), 6.20-6.75 (m, 6H, aromatic), 7.12-7.84 (m, 4H, aromatic) ppm. Mass spectra: m/z 418 (M⁺).

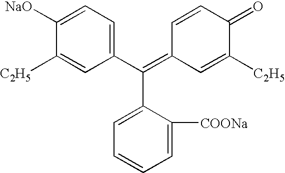

### Example 19

### Synthesis of disodium salt of 3,3-bis-(4-hydroxy-3-isopropylphenyl)-1-(3H)-isobenzofuranone

A mixture of 3,3-bis-(4-hydroxy-3-isopropylphenyl)-1-(3H)-isobenzofuranone (0.01 mol) in ethanol (50 mL, 85%) was stirred followed by addition of sodium hydroxide (0.02 mol) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 81% yield. ¹H-NMR (DMSO-d₆): δ 1.02-1.14 (dd, 12H, 4CH₃), 3.12-3.45 (heptate, 2H, 2CH), 6.32-6.76 (m, 6H, aromatic), 7.30-7.83 (m, 4H, aromatic) ppm. Mass spectra: m/z 446 (M⁺).

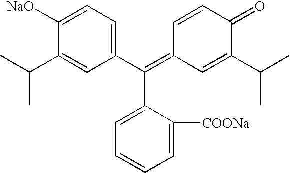

### Example 20

### Synthesis of disodium salt of 3,3-bis-(4-hydroxy-3-methoxyphenyl)-1-(3H)-isobenzofuranone

A mixture of 3,3-bis-(4-hydroxy-3-methoxyphenyl)-1-(3H)-isobenzofuranone (0.01 mol) in ethanol (50 mL, 85%) was stirred followed by addition of sodium hydroxide (0.02 mol) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 88% yield.

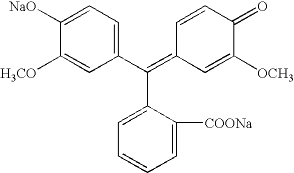

### Example 21

### Synthesis of disodium salt of 3,3-bis-(4-hydroxy-2,3,5-trimethylphenyl)-1-(3H)-isobenzofuranone

A mixture of 3,3-bis-(4-hydroxy-2,3,5-trimethylphenyl)-1-(3H)-isobenzofuranone (0.01 mol) in ethanol (50 mL, 85%) was stirred followed by addition of sodium hydroxide (0.02 mol) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 80% yield.

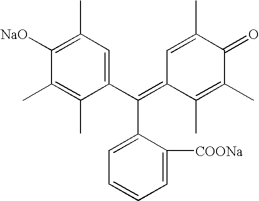

### Example 22

### Synthesis of disodium salt of 3,3-bis-(4-hydroxy-3-sec-butylphenyl)-1-(3H)-isobenzofuranone

A mixture of 3,3-bis-(4-hydroxy-3-sec-butylphenyl)-1-(3H)-isobenzofuranone (0.01 mol) in ethanol (50 mL, 85%) was stirred followed by addition of sodium hydroxide (0.02 mol) in ethanol (50 mL, 85%). The reaction mixture was stirred and refluxed for 2 hours, cooled to room temperature. The 5 solvent was evaporated on rotary evaporator, isolated the crude product and dried. Recrystallization from ethanol furnished pure disodium salt in 82% yield. ¹H-NMR (DMSO-d₆): δ 0.80 (t, 6H, 2CH₃), 1.29-1.37 (p, 4H, 2CH₂), 1.20 (d, 6H, 2CH₃), 2.88-2.96 (sextate, 2H, 2CH), 6.08-6.75 (m, 6H, aromatic), 7.37-7.81 (m, 4H, aromatic) ppm.

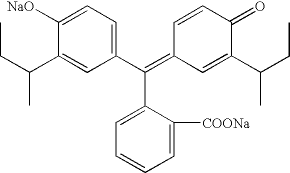

### Example 23

### Synthesis of disodium salt of 3,3-bis-(4-hydroxy-3-nitrophenyl)-1-(3H)-isobenzofuranone

A mixture of 3,3-bis-(4-hydroxy-3-nitrophenyl)-1-(3H)-isobenzofuranone (0.01 mol) in ethanol (50 mL, 85%) was stirred followed by addition of sodium hydroxide (0.02 mol) in ethanol (50 mL, 85%). The reaction mixture was stirred and refluxed for 2 hours, cooled to room temperature. The solvent was evaporated on rotary evaporator, isolated the crude product and dried. Recrystallization from ethanol furnished pure disodium salt in 92% yield. 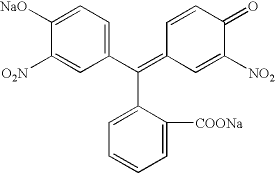
Synthesis of Phenol and Hydrazine Acid-Base Indicators

### Example 1

### Synthesis of sodium salt of 5-methyl-2-nitrophenol

A mixture of 5-methyl-2-nitrophenol (0.1M) in ethanol (25 mL, 85%) was stirred followed by addition of sodium hydroxide (0.1M) in ethanol (25 mL, 85%). The reaction mixture was stirred at room temperature for 2 hours. The separated golden yellow solid was filtered, washed with ethanol and dried. Recrystallization from ethanol furnished pure sodium salt in 96% yield.

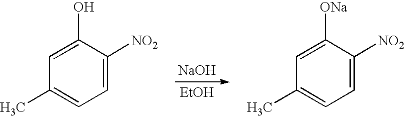

### Example 2

### Synthesis of p-nitrobenzhydrazide

A mixture of ethyl p-nitrobenzoate (0.1M), hydrazine hydrate (0.11M) in ethanol (100 mL) was stirred at room temperature for 2 hours. The separated pale yellow solid was filtered, washed with ethanol and dried. Recrystallization from ethanol furnished pure hydrazide in 88% yield.

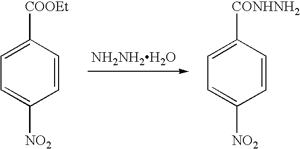

### Example 3

### Synthesis of hydrazide

A mixture of ethyl salicylate (0.1M), 2,4-dinitrophenylhydrazine (0.1M) in ethanol (150 mL) was stirred at room temperature for 2 hours. The separated orange solid was filtered, washed with ethanol and dried. Recrystallization from ethanol furnished pure hydrazide in 94% yield.

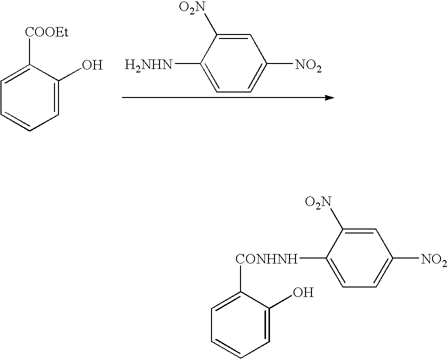

### Example 4

### Synthesis of sodium salt of hydrazide

A mixture of hydrazide (0.1M) in ethanol (25 mL) was stirred followed by addition of sodium ethoxide (0.1M) in ethanol (25 mL). The reaction mixture was stirred and refluxed at for 4 hours, cooled to room temperature. The solvent was evaporated on rotary evaporator, isolated the crude yellow product and dried. Recrystallization from ethanol furnished pure sodium salt in 92% yield.

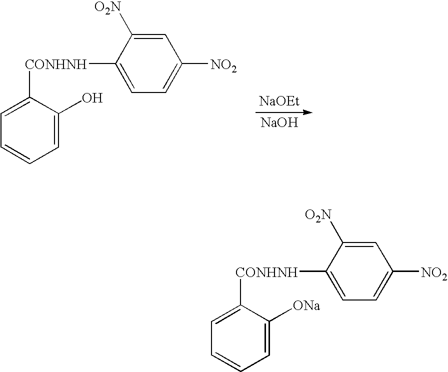

### Example 5

### Synthesis of hydrazide

A mixture of ethyl salicylate (0.1M), 4-nitrophenylhydrazine (0.1M) in ethanol (150 mL) was stirred at room temperature for 2 hours. The separated yellow solid was filtered, washed with ethanol and dried. Recrystallization from ethanol furnished pure hydrazide in 89% yield.

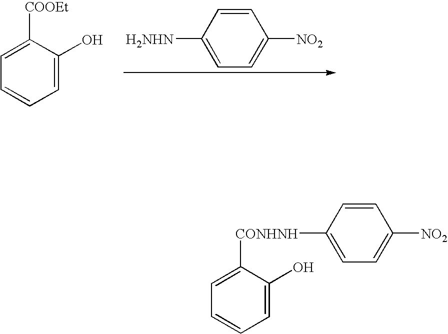

### Example 6

### Synthesis of sodium salt of hydrazide

A mixture of hydrazide (0.1M) in ethanol (25 mL) was stirred followed by addition of sodium ethoxide (0.1M) in ethanol (25 mL). The reaction mixture was stirred and refluxed at for 4 hours, cooled to room temperature. The solvent was evaporated on rotary evaporator, isolated the crude yellow product and dried. Recrystallization from ethanol furnished pure sodium salt in 84% yield.

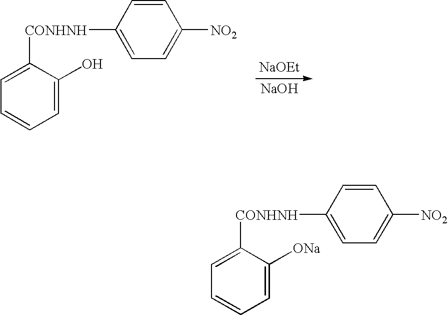

As various changes could be made in the above products, without departing from the scope of the invention, it is intended that all matter contained in the above description shall be interpreted as illustrative and not in a limiting sense.

Although the present invention has been described with reference to preferred embodiments, persons skilled in the art will recognize that changes may be made in form and detail without departing from the spirit and scope of the invention.

Those skilled in the art will recognize, or be able to ascertain, using no more than routine experimentation, many equivalents to specific embodiments of the invention described specifically herein. Such equivalents are intended to be encompassed in the scope of the following claim.

What is claimed is:

1. A toothpaste comprising:
a surfactant; and
an acid-base indicator comprising: 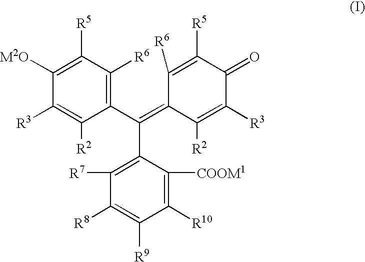
wherein
R² is selected from the group consisting of hydrogen, nitro, amino and alkyl;
R³ is selected from the group consisting of hydrogen, aryl, alkyl, nitro, acetamido and alkoxide;
R⁵ is selected from the group consisting of hydrogen, halo, alkoxide and alkyl;
R⁶ is selected from the group consisting of hydrogen and alkyl;
R⁷, R⁸, R⁹ and R¹⁰ are all hydrogen;
optionally, one of the carbons connected to R², R³, R⁵ or R⁶ can be substituted with a nitrogen atom;
M¹ and M² are each independently a hydrogen atom, a metal ion or an ammonium ion, provided that at least one of M¹ or M² is a metal ion or an ammonium ion;
a gelling agent; and
a polishing agent.

2. The toothpaste of claim 1, wherein wherein R² is selected from the group consisting of hydrogen and methyl; R³ is selected from the group consisting of hydrogen, phenyl, isopropyl, methyl, ethyl, sec-butyl, nitro and methoxy; R⁵ is selected from the group consisting of hydrogen, bromo, methoxy, isopropyl and methyl; and R⁶ is selected from the group consisting of hydrogen and methyl.

3. The toothpaste of claim 1, wherein R² is hydrogen, R³ is Me, and R⁵, R⁶, R⁷, R⁸, R⁹ and R¹⁰ are hydrogen atoms, or R² is Me, R³is a hydrogen atom, R⁵ is an iso-propyl group, R⁶, R⁷, R⁸, R⁹ and R¹⁰ are hydrogen atoms or R² is H, R³ is Me, R⁵ is Br and R⁶, R⁷, R⁸, R⁹ and R¹⁰ are hydrogen atoms or R² is Me, R³is Br, R⁵ is an isopropyl and R⁶, R⁷, R⁸, R⁹ and R¹⁰ are hydrogen atoms.

4. The toothpaste of claim 1, wherein R² is H, R³is phenyl and R⁵, R⁶, R⁷, R⁸, R⁹ and R¹⁰ are all hydrogen atoms, or R² is H, R³ and R⁵ are isopropyl and R⁶, R⁷, R⁸, R⁹ and R¹⁰ are all hydrogen atoms, or R² is H, R³ is methyl, R⁵ is H, R⁶is methyl, R⁷, R⁸, R⁹ and R¹⁰ are all hydrogen atoms, or R² is H, R³ and R⁵ are methoxy and R⁶, R⁷, R⁸, R⁹ and R¹⁰ are all hydrogen atoms, or R² is H, R³ and R⁵ are methyl and R⁶, R⁷, R⁸, R⁹ and R¹⁰ are all hydrogen atoms, or R² is H, R³ is ethyl and R⁵, R⁶, R⁷, R⁸, R⁹ and R¹⁰ are all hydrogen atoms, or R² is H, R³is isopropyl and R⁵, R⁶, R⁷, R⁸, R⁹ and R¹⁰ are all hydrogen atoms, or R² is H, R³is methoxide and R⁵, R⁶, R⁷, R⁸, R⁹ and R¹⁰ are all hydrogen atoms or R² is H, R³ and R⁵ are all methyl and R⁶, R⁷, R⁸, R⁹ and R¹⁰ are all hydrogen atoms, or R², R³, R⁵, R⁶, R⁷, R⁸, R⁹, R¹⁰ are all hydrogen atoms and R³ is sec-butyl, or R², R³, R⁵, R⁶, R⁷, R⁸, R⁹, R¹⁰ are all hydrogen atoms and R³ is nitro.

5. The toothpaste of claim 1, further comprising a base.

6. The toothpaste of claim 5, wherein the base is a metal hydroxide.

7. The toothpaste of claim 1, wherein said polishing agent is selected from the group consisting of silica gel, silica, sodium aluminum silicate, hydrated alumina, dicalcium phosphate, calcium pyrophosphate, calcium carbonate and mixtures thereof.

8. The toothpaste of claim 1, wherein from approximately 0.5 to approximately 18% by weight of a gelling agent is incorporated therein.

9. The toothpaste of claim 8, wherein said gelling agent is selected from the group consisting of sodium carboxymethyl cellulose, xanthan gum, polyvinyl pyrrolidone, hydroxyethyl cellulose, polyvinyl alcohol, Irish moss extract, sodium alginate and mixtures thereof.

10. A method to indicate when brushing of the teeth is complete, comprising the step of:
applying a toothpaste composition to a tooth comprising
a surfactant; and
an acid-base indicator comprising: 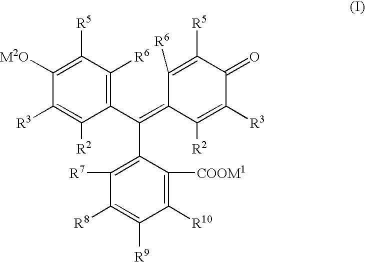
wherein
R² is selected from the group consisting of hydrogen, nitro, amino and alkyl;
R³ is selected from the group consisting of hydrogen, aryl, alkyl, nitro, acetamido and alkoxide;
R⁵ is selected from the group consisting of hydrogen, halo, alkoxide and alkyl;
R⁶ is selected from the group consisting of hydrogen and alkyl;
R⁷, R⁸, R⁹ and R¹⁰ are all hydrogen;
optionally, one of the carbons connected to R², R³, R⁵ or R⁶ can be substituted with a nitrogen atom;
M¹ and M² are each independently a hydrogen atom, a metal ion or an ammonium ion, provided that at least one of M¹ or M² is a metal ion or an ammonium ion;
a gelling agent;
a polishing agent; and
brushing the composition for a period of time sufficient such that the color of the indicator changes.

11. The method of claim 10, wherein R² is selected from the group consisting of hydrogen and methyl; R³ is selected from the group consisting of hydrogen, phenyl, isopropyl, methyl, ethyl, sec-butyl, nitro and methoxy; R⁵ is selected from the group consisting of hydrogen, bromo, methoxy, isopropyl and methyl; and R⁶ is selected from the group consisting of hydrogen and methyl.

12. The method of claim 10, wherein R² is hydrogen, R³ is Me, and R⁵, R⁶, R⁷, R⁸, R⁹ and R¹⁰ are hydrogen atoms, or R² is Me, R³ is a hydrogen atom, R⁵ is an iso-propyl group, R⁶, R⁷, R⁸, R⁹ and R¹⁰ are hydrogen atoms or R² is H, R³ is Me, R⁵ is Br and R⁶, R⁷, R⁸, R⁹ and R¹⁰ are hydrogen atoms or R² is Me, R³ is Br, R⁵ is an isopropyl and R⁶, R⁷, R⁸, R⁹ and R¹⁰ are hydrogen atoms.

13. The method of claim 10, wherein R² is H, R³ is phenyl and R⁵, R⁶, R⁷, R⁸, R⁹ and R¹⁰ are all hydrogen atoms, or R² is H, R³ and R⁵ are isopropyl and R⁶, R⁷, R⁸, R⁹ and R¹⁰ are all hydrogen atoms, or R² is H, R³is methyl, R⁵ is H, R⁶ is methyl, R⁷, R⁸, R⁹ and R¹⁰ are all hydrogen atoms, or R² is H, R³ and R⁵ are methoxy and R⁶, R⁷, R⁸, R⁹ and R¹⁰ are all hydrogen atoms, or R² is H, R³ and R⁵ are methyl and R⁶, R⁷, R⁸, R⁹ and R¹⁰ are all hydrogen atoms, or R² is H, R³ is ethyl and R⁵, R⁶, R⁷, R⁸, R⁹ and R¹⁰ are all hydrogen atoms, or R² is H, R³ is isopropyl and R⁵, R⁶, R⁷, R⁸, R⁹ and R¹⁰ are all hydrogen atoms, or R² is H, R³ is methoxide and R⁵, R⁶, R⁷, R⁸, R⁹ and R¹⁰ are all hydrogen atoms or R², R³ and R⁵ are all methyl and R⁶, R⁷, R⁸, R⁹ and R¹⁰ are all hydrogen atoms, or R², R³, R⁵, R⁶, R⁷, R⁸, R⁹, R¹⁰ are all hydrogen atoms and R³ is sec-butyl, or R², R³, R⁵, R⁶, R⁷, R⁸, R⁹, R¹⁰ are all hydrogen atoms and R³ is nitro.

14. The method of claim 10, further comprising a base.

15. The method of claim 14, wherein the base is a metal hydroxide.

16. The method of claim 10 wherein said polishing agent is selected from the group consisting of silica gel, silica, sodium aluminum silicate, hydrated alumina, dicalcium phosphate, calcium pyrophosphate, calcium carbonate and mixtures thereof.

17. The method of claim 10, wherein from approximately 0.5 to approximately 18% by weight of a gelling agent is incorporated therein.

18. The method of claim 17, wherein said gelling agent is selected from the group consisting of sodium carboxymethyl cellulose, xanthan gum, polyvinyl pyrrolidone, hydroxyethyl cellulose, polyvinyl alcohol, Irish moss extract, sodium alginate and mixtures thereof.

19. A mouthwash composition comprising:
a surfactant; and
an acid-base indicator comprising: 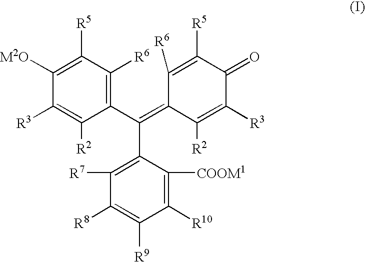
wherein
R² is selected from the group consisting of hydrogen, nitro, amino and alkyl;
R³ is selected from the group consisting of hydrogen, aryl, alkyl, nitro, acetamido and alkoxide;
R⁵ is selected from the group consisting of hydrogen, halo, alkoxide and alkyl;
R⁶ is selected from the group consisting of hydrogen and alkyl;
R⁷, R⁸, R⁹ and R¹⁰ are all hydrogen;
optionally, one of the carbons connected to R², R³, R⁵ or R⁶ can be substituted with a nitrogen atom;
M¹ and M² are each independently a hydrogen atom, a metal ion or an ammonium ion, provided that at least one of M¹ or M² is a metal ion or an ammonium ion; and
a humectant.

20. The mouthwash of claim 19, wherein R²is selected from the group consisting of hydrogen and methyl; R³ is selected from the group consisting of hydrogen, phenyl, isopropyl, methyl, ethyl, sec-butyl, nitro and methoxy; R⁵ is selected from the group consisting of hydrogen, bromo, methoxy, isopropyl and methyl; and R⁶ is selected from the group consisting of hydrogen and methyl.

21. The mouthwash of claim 19, wherein R² is hydrogen, R³is Me, and R⁵, R⁶, R⁷, R⁸, R⁹ and R¹⁰ are hydrogen atoms, or R² is Me, R³ is a hydrogen atom, R⁵ is an iso-propyl group, R⁶, R⁷, R⁸, R⁹ and R¹⁰ are hydrogen atoms or R² is H, R³is Me, R⁵ is Br and R⁶, R⁷, R⁸, R⁹ and R¹⁰ are hydrogen atoms or R² is Me, R³is Br, R⁵ is an isopropyl and R⁶, R⁷, R⁸, R⁹ and R¹⁰ are hydrogen atoms.

22. The mouthwash of claim 19, wherein R² is H, R³is phenyl and R⁵, R⁶, R⁷, R⁸, R⁹ and R¹⁰ are all hydrogen atoms, or R² is H, R³ and R⁵ are isopropyl and R⁶, R⁷, R⁸, R⁹ and R¹⁰ are all hydrogen atoms, or R² is H, R³ is methyl, R⁵ is H, R⁶ is methyl, R⁷, R⁸, R⁹ and R¹⁰ are all hydrogen atoms, or R² is H, R³ and R⁵ are methoxy and R⁶, R⁷, R⁸, R⁹ and R¹⁰ are all hydrogen atoms, or R² is H, R³ and R⁵ and methyl and R⁶, R⁷, R⁸, R⁹ and R¹⁰ are all hydrogen atoms, or R² is H, R³ is ethyl and R⁵, R⁶, R⁷, R⁸, R⁹ and R¹⁰ are all hydrogen atoms, or R² is H, R³ is isopropyl and R⁵, R⁶, R⁷, R⁸, R⁹ and R¹⁰ are all hydrogen atoms, or R² is H, R³ is methoxide and R⁵, R⁶, R⁷, R⁸, R⁹ and R¹⁰ are all hydrogen atoms, or R², R³ and R⁵ are all methyl and R⁶, R⁷, R⁸, R⁹ and R¹⁰ are all hydrogen atoms, or R², R³, R⁵, R⁶, R⁷, R⁸, R⁹, R¹⁰ are all hydrogen atoms and R³ is sec-butyl, or R², R³, R⁵, R⁶, R⁷, R⁸, R⁹, R¹⁰ are all hydrogen atoms and R³ is nitro.

23. The mouthwash of claim 19, further comprising a base.

24. The mouthwash of claim 23, wherein the base is a metal hydroxide.

25. The mouthwash of claim 19, wherein the humectant is glycerine.

26. A method to indicate when treatment of the oral cavity is complete, comprising the step of:
contacting a mouthwash composition to the oral cavity comprising
a surfactant; and
an acid-base indicator comprising: 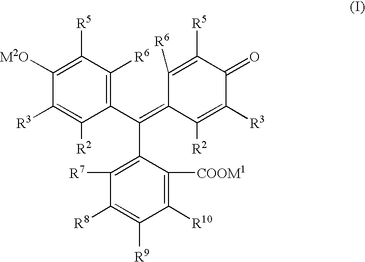
wherein
R² is selected from the group consisting of hydrogen, nitro, amino and alkyl;
R³ is selected from the group consisting of hydrogen, aryl, alkyl, nitro, acetamido and alkoxide;
R⁶ is selected from the group consisting of hydrogen, halo, alkoxide and alkyl;
R⁶ is selected from the group consisting of hydrogen and alkyl;
R⁷, R⁸, R⁹ and R¹⁰ are all hydrogen;
optionally, one of the carbons connected to R², R³, R⁵ or R⁶ can be substituted with a nitrogen atom;
M¹ and M² are each independently a hydrogen atom, a metal ion or an ammonium ion, provided that at least one of M¹ or M² is a metal ion or an ammonium ion;
a humectant; and
contacting the oral cavity for a period of time sufficient such that the color of the indicator changes.

27. The method of claim 26, wherein R² is selected from the group consisting of hydrogen and methyl; R³ is selected from the group consisting of hydrogen, phenyl, isopropyl, methyl, ethyl, sec-butyl, nitro and methoxy; R⁵ is selected from the group consisting of hydrogen, bromo, methoxy, isopropyl and methyl; and R⁶ is selected from the group consisting of hydrogen and methyl.

28. The method of claim 26, wherein R² is hydrogen, R³is Me, and R⁵, R⁶, R⁷, R⁸, R⁹ and R¹⁰ are hydrogen atoms, or R² is Me, R³ is a hydrogen atom, R⁵ is an iso-propyl group, R⁶, R⁷, R⁸, R⁹ and R¹⁰ are hydrogen atoms or R² is H, R³ is Me, R⁵ is Br and R⁶, R⁷, R⁸, R⁹ and R¹⁰ are hydrogen atoms or R² is Me, R³is Br, R⁵ is an isopropyl and R⁶, R⁷, R⁸, R⁹ and R¹⁰ are hydrogen atoms.

29. The method of claim 26, wherein R² is H, R³ is phenyl and R⁵, R⁶, R⁷, R⁸, R⁹ and R¹⁰ are all hydrogen atoms, or R² is H, R³ and R⁵ are isopropyl and R⁶, R⁷, R⁸, R⁹ and R¹⁰ are all hydrogen atoms, or R² is H, R³ is methyl, R⁵ is H, R⁶ is methyl, R⁷, R⁸, R⁹ and R¹⁰ are all hydrogen atoms, or R² is H, R³ and R⁵ are methoxy and R⁶, R⁷, R⁸, R⁹ and R¹⁰ are all hydrogen atoms, or R² is H, R³ and R⁵ are methyl and R⁶, R⁷, R⁸, R⁹ and R¹⁰ are all hydrogen atoms, or R² is H, R³ is ethyl and R⁵, R⁶, R⁷, R⁸, R⁹ and R¹⁰ are all hydrogen atoms, or R² is H, R³ is isopropyl and R⁵, R⁶, R⁷, R⁸, R⁹ and R¹⁰ are all hydrogen atoms, or R² is H, R³ is methoxide and R⁵, R⁶, R⁷, R⁸, R⁹ and R¹⁰ are all hydrogen atoms, or R², R³ and R⁵ are all methyl and R⁶, R⁷, R⁸, R⁹ and R¹⁰ are all hydrogen atoms, or R², R³, R⁵, R⁶, R⁷, R⁸, R⁹, R¹⁰ are all hydrogen atoms and R³is sec-butyl, or R², R³, R⁵, R⁶, R⁷, R⁸, R⁹, R¹⁰, are all hydrogen atoms and R³is nitro.

30. The method of claim 26, further comprising a base.

31. The method of claim 30, wherein the base is a metal hydroxide.

32. The method of claim 26, wherein the humectant is glycerine.
